(19) United States
(12) Patent Application Publication
(10) Pub. No.: US 2023/0121528 A1
(43) Pub. Date: Apr. 20, 2023

(54) SALMONELLA-BASED DNA VACCINES IN COMBINATION WITH AN ANTIBIOTIC

(71) Applicant: VAXIMM AG, Basel (CH)

(72) Inventor: Heinz LUBENAU, Neustadt an der Weinstrasse (DE)

(21) Appl. No.: 17/791,282

(22) PCT Filed: Jan. 12, 2021
(86) PCT No.: PCT/EP2021/050470

§ 371 Date: Jul. 7, 2022

(30) Foreign Application Priority Data
Jan. 13, 2020 (EP) 20151519.4
May 12, 2020 (EP) 20174083.4

(51) Int. Cl. A61K 39/00; A61K 39/395; A61K 31/635; A61K 31/505; A61P 35/00

(52) U.S. Cl. CPC A61K 39/001182; A61K 31/505; A61K 31/635; A61K 39/0011; A61K 39/001109; A61K 39/001153; A61K 39/001168; A61K 39/3955; A61P 35/00; A61K 2039/70; A61K 2039/523; A61K 2039/542

(57) ABSTRACT

The present invention relates to a Salmonella typhi Ty21a strain comprising a DNA molecule comprising at least one eukaryotic expression cassette encoding at least one tumor antigen, stroma antigen and/or checkpoint inhibitor antigen for the use in the treatment of cancer in a human subject following treatment with an antibiotic, wherein the Salmonellatyphi Ty21a strain is to be administered orally and optionally in combination with a checkpoint inhibitor.

### FIELD OF THE INVENTION

The present invention relates to a Salmonella typhi Ty21a strain comprising a DNA molecule comprising at least one eukaryotic expression cassette encoding at least one tumor antigen, stroma antigen and/or checkpoint inhibitor antigen for the use in the treatment of cancer in a human subject following treatment with an antibiotic, wherein the Salmonella typhi Ty21a strain is to be administered orally and optionally in combination with a checkpoint inhibitor.

### BACKGROUND OF THE INVENTION

The finding that tumors can be immunogenic has led to the development of a number of cancer immunotherapies designed to employ the immune system to selectively eliminate malignant cells while sparing normal tissue. However, survival benefits from vaccination against tumor antigens alone remain modest. Anti-cancer vaccines face numerous challenges, including the immunosuppressive microenvironment and an optimal immune stimulation against a host protein without eliciting autoimmune reactions. The abnormal tumor vasculature creates a hypoxic microenvironment that polarizes inflammatory cells toward immune suppression. Moreover, tumors systemically alter immune cells' proliferation, differentiation, and function via secretion of growth factors and cytokines.

While there are several ways of immunizing against cancer a very promising way is the use of bacteria such as Salmonella as carrier for a DNA vaccine against a tumor antigen or stroma antigen. For example, WO 2014/005683 discloses an attenuated strain of Salmonella comprising a recombinant DNA molecule encoding a VEGF receptor protein for use in cancer immunotherapy, particularly for use in the treatment of pancreatic cancer. This vaccine expressing human VEGFR-2 is also referred to as VXM01.

Further, WO 2014/173542, WO 2015/090584, WO 2016/2020458 and WO 2018/167290 disclose an attenuated strain of Salmonella comprising a recombinant DNA molecule encoding Wilms' tumor protein, mesothelin, CMVpp65 or PD-L1, respectively, for use in cancer immunotherapy.

WO 2013/09189 discloses a method for growing attenuated mutant Salmonella typhi strains lacking galactose epimerase activity and harboring a recombinant DNA molecule and WO 2018/011289 discloses a fast and effective method of generating personalized cancer vaccines comprising an attenuated strain of Salmonella.

Glioblastoma is the most aggressive cancer that begins within the brain and the WHO grade IV is the most aggressive form of gliomas. Patient median survival after first diagnosis is still below 15 months in study cohorts, nearly all patients suffer from tumor recurrence, and only 25% survive more than 1 year. Since 2005 surgery followed by radiotherapy in combination with temozolomide serves as the standard first line treatment in glioblastoma. After failure of initial treatment further therapeutic options are limited. There is no standard treatment for recurrent glioblastoma. New and more effective immunotherapeutic approaches are highly needed to increase patients' survival. VXM01 is a VEGFR-2 coding DNA vaccine, using a Salmonella Ty21a carrier for oral administration. High expression of VEGFR-2 on glioblastoma tumor tissue and tumor vasculature serves as a promising target for VEGFR-2 primed T cells. In a phase I/II VXM01 study in glioblastoma administration of VXM01 in 14 recurrent tumor patients showed an acceptable safety profile. Objective clinical responses in 2 patients (CR and PR) and prolonged overall survival could be associated with the VEGFR-2-specific immune response, J Clin Oncol 36, 2018 (suppl; abstr. 2017).

While therapeutic options in recurrent glioblastoma are particularly limited and prognosis for patients having this particular solid tumor is poor, there exists a general need for improved cancer therapy approaches, particularly in further improving therapeutic vaccinations against tumors, including combination therapies. Checkpoint inhibitors have previously been described to improve vaccination with Salmonella-based DNA vaccines, such as in WO 2016/202459 and WO 2018/083209.

The microbiota, i.e., the resident microbes of a host, has recently gained interest. The intestinal microbiome, in particular, hosts an abundance and great diversity of microbes that perform a range of essential and beneficial functions, including the metabolism of nutrients, the maintenance of gut homeostasis and the regulation of gut mucosal immunity. Given the growing list of ways that the microbiome can influence the immune system, it would be surprising if the microbiome did not also influence vaccine responses, however, evidence that this is the case is, to date, relatively limited (Lynn and Pulendran, J. Leukoc. Biol., 2018; 103(2): 225-231). The microbiota has been speculated to act as a vaccine adjuvant and to be important for antibody responses, but its interaction and effects is far from being understood. Even less is known about T cell responses, which are critical for therapeutic vaccines against solid tumors. Also, very little is known about the role of the microbiome in immune responses in humans.

In one study a positive effect of antibiotics on B cell responses to vaccines, including live-attenuated Salmonella typhi Ty21a has been reported in mice (Woo et al., Clinical and Diagnostic Laboratory Immunology, 1999; 6(6): 832-837). In this study Salmonella typhi Ty21a transformed with pBR322 to make it ampicillin and doxycycline resistant and intrinsically resistant to clarithromycin was administered intraperitoneally concomitantly with ampicillin, doxycycline or clarithromycin to mice. No effects on T cell responses have been reported.

The inventors have surprisingly found that pre-treatment with antibiotics enhance the therapeutic effect of oral Salmonella-based DNA vaccines against cancer in humans.

### SUMMARY OF THE INVENTION

The present invention relates to a Salmonella typhi Ty21a strain comprising a DNA molecule comprising at least one eukaryotic expression cassette encoding at least one tumor antigen, stroma antigen and/or checkpoint inhibitor antigen for the use in the treatment of cancer in a human subject following treatment with an antibiotic, wherein the Salmonella typhi Ty21a strain is to be administered orally.

In one embodiment the Salmonella typhi Ty21a strain comprises a DNA molecule comprising at least one eukaryotic expression cassette encoding at least one antigen selected from the group consisting of human Wilms’ Tumor protein (WT1), human Mesothelin (MSLN), human CEA, CMV pp65, human PD-L1, human VEGFR-2 and human fibroblast activation protein (FAP). In another embodiment the Salmonella typhi Ty21a strain comprises a DNA molecule comprising at least one eukaryotic expression cassette encoding at least one neoantigen, preferably at least one eukaryotic expression cassette encoding at least one polypeptide comprising five or more neoantigens.

Optionally the Salmonella typhi Ty21a strain may be administered in combination with at least one checkpoint inhibitor, preferably simultaneously with or prior to said at least one checkpoint inhibitor. In one embodiment the Salmonella typhi Ty21a strain is to be administered in combination with at least one checkpoint inhibitor, wherein the at least one checkpoint inhibitor is preferably an immunomodulatory antibody selected from the group consisting of antibodies against PD-1, PD-L1, CTLA-4, IDO, GITR, OX40, TIM-3, LAG-3, KIR, CSF1R and CD137.

In certain embodiments the Salmonella typhi Ty21a strain is to be administered at least 3 days after completion of the treatment with the antibiotic. In certain embodiments the Salmonella typhi Ty21a strain is to be administered within 1 month of completion of the treatment with the antibiotic, preferably the first dose of the Salmonella typhi Ty21a strain is to be administered between about at least 3 days to 1 month. Preferably, the antibiotic is an antibiotic the Salmonella typhi Ty21a strain comprising a DNA molecule comprising at least one eukaryotic expression cassette encoding at least one tumor antigen, stroma antigen and/or checkpoint inhibitor antigen is not resistant to.

The antibiotic to be administered may be a combination preparation. In certain embodiments the antibiotic is selected from the group consisting of a penicillin (e.g. amoxicillin, ampicillin, piperacillin or flucloxacillin), a cephalosporin, a polymyxin (e.g. colistin), a rifamycin (e.g. rifaximin), a lipiarmycin, a quinolone (e.g. ciprofloxacin), a sulfonamide (e.g. sulfamethoxazole), a macrolide (erythromycin), a linocosamide, a tetracycline (e.g. tetracycline), an aminoglycoside (e.g. paromomycin), a cyclic lipopeptide (e.g. daptomycin), a glycylcycline (e.g. tigecycline), an oxozolidinone (e.g. linezolid), a nitrodimazole (e.g. metronidazole), a lipiarmycin (e.g. fidaxomicin) and a dihydrofolate reductase inhibitor (e.g. a diaminopyrimidine, such as trimethoprim or tetroxoprim). In one embodiment the antibiotic is sulfamethoxazole or trimethoprim or a combination thereof. In another embodiment the antibiotic is cotrimoxazol.

The Salmonella typhi Ty21a strain for use according to invention may also be accompanied by chemotherapy or radiotherapy.

In preferred embodiments the cancer to be treated is a solid tumor, such as colorectal cancer, pancreatic cancer, lung cancer, ovarian cancer, mesothelioma, glioblastoma, gastric cancer, hepatocellular cancer, renal cell cancer, prostate cancer, cervical cancer, breast cancer and melanoma. In one embodiment the solid tumor is a glioblastoma, preferably recurrent glioblastoma.

The Salmonella typhi Ty21a strain for use according to the invention may be administered at a single dose of the Salmonella typhi Ty21a strain comprising from about 10⁶ to about 10⁹, more particular from about 10⁶ to about 10⁸, most particular from about 10⁶ to about 10⁷ colony forming units (CFU); and/or wherein the Salmonella typhi Ty21a strain is to be administered 2 to 4 times in the first week, followed by a single dose boosting administration every 2 to 4 weeks. The Salmonella typhi Ty21a strain may be in the form of a pharmaceutical composition, further comprising at least one pharmaceutically acceptable excipient.

### SHORT DESCRIPTIONS OF THE FIGURES

FIG. 1 shows the plasmid map of pVAX10.VR2-1 expressing as an exemplary antigen VEGFR-2.

FIG. 2A shows a schematic overview of the phase I/II combination clinical trial in patients with recurrent glioblastoma treated with VXM01 and anti-PD-L1 checkpoint inhibitor avelumab including the time line of the clinical trial and the individual responses of the participating patients, such as partial responses (PR), stable disease (SD) and progressive disease (PD).

FIG. 2B shows a schematic overview of the phase I/II combination clinical trial in patients with recurrent glioblastoma treated with VXM01 and anti-PD-L1 checkpoint inhibitor avelumab co-treated with antibiotics including the time line of the clinical trial and the individual responses of the participating patients, such as partial responses (PR), stable disease (SD) and progressive disease (PD). The treatment period of those 4 patients with Cotrim forte® is indicated by a black bar.

FIG. 3 shows the tumor response of 9 patients treated with VXM01 and anti-PD-L1 checkpoint inhibitor avelumab as indicated in FIG. 2. The individual patient number is provided on the x-axis and the tumor diameter as a percentage of the tumor diameter at baseline (d0) is given on the y-axis at the month indicated.

FIG. 4 shows the VEGFR-2 specific T cell response and the tumor response of the treatment with VXM01 and avelumab in patient No. 0104, pretreated with Cotrim forte®. A) Results of an Enzyme Linked Immuno Spot Assay (ELISpot) in blood samples of patient No. 0104 on day 0 of the clinical trial and after about 3, 6 and 9 months of the clinical trial are shown (pool minus negative control) as VEGFR-2-pool specific spot counts per 4×10⁵ peripheral blood mononuclear cells (PBMCs). B) The change in tumor volume relative to baseline after 3, 6 and 9 months of the clinical trial in patient No. 0104 is shown.

FIG. 5 shows the level of intra-tumoral immune biomarkers in immunohistochemistry sections of tumor samples obtained at base line from patient No. 0104. A) Levels of CD8+ T cells, FoxP3+ T cells and CD68+ T cells per mm² are shown. B) PD-1 and PD-L1 staining is shown as histo Score.

FIG. 6 shows the level of intra-tumoral immune biomarkers at baseline and the tumor response following treatment with VXM01 and avelumab in patient No. 0109, pretreated with Cotrim forte® from study day 3 to study day 7, i.e. covering the time points of the 2^{nd} to 4^{th} initial study drug administration. A) Levels of CD8+ T cells, FoxP3+ T cells and CD68+ T cells per mm² at baseline are shown. B) PD-1 and PD-L1 staining at baseline is shown as histo Score.

FIG. 7 shows the tumor response in 3 evaluable patients treated with VXM01 and the anti-PD-1 checkpoint inhibitor Nivolumab in a previous clinical study. The tumor size decreased in patient No 2611 and 2603 indicating PR after 30 and CR after 6 months of treatment with VXM01 and Nivolumab, respectively. In patient 2603 a partial response (with a tumor size reduction from 1511 mm at baseline to 2×2 mm at month 3) has already been observed at months 3 with VXM01 monotherapy.

### DETAILED DESCRIPTION OF THE INVENTION

In one aspect the invention relates to a Salmonella typhi Ty21a strain comprising a DNA molecule comprising at least one eukaryotic expression cassette encoding at least one tumor antigen, stroma antigen and/or checkpoint inhibitor antigen for the use in the treatment of cancer in a human subject following treatment with an antibiotic, wherein the Salmonella typhi Ty21a strain is to be administered orally.

In another aspect the invention relates to a method for treating cancer in a human subject comprising administering an antibiotic to said human subject followed by oral administration of a Salmonella typhi Ty21a strain comprising a DNA molecule comprising at least one eukaryotic expression cassette encoding at least one tumor antigen, stroma antigen and/or checkpoint inhibitor antigen.

In yet another aspect the invention relates to a Salmonella typhi Ty21a strain comprising a DNA molecule comprising at least one eukaryotic expression cassette encoding at least one tumor antigen, stroma antigen and/or checkpoint inhibitor antigen for the use in the treatment of cancer in a human subject, wherein the subject has been or is treated with at least one antibiotic and the Salmonella typhi Ty21a strain is to be administered orally.

A tumor antigen may be a tumor-specific antigen, or a tumor-associated antigen. Tumor-specific antigens encompass neoantigens. Thus, in certain embodiments the Salmonella typhi Ty21a strain comprises a DNA molecule comprising at least one eukaryotic expression cassette encoding at least one neoantigen. Alternatively, the Salmonella typhi Ty21a strain may be specified to comprise a DNA molecule comprising at least one eukaryotic expression cassette encoding at least one tumor antigen, neoantigen, stroma antigen and/or checkpoint inhibitor antigen throughout the application. In one embodiment the Salmonella typhi Ty21a strain comprises a DNA molecule comprising at least one eukaryotic expression cassette encoding at least one neoantigen, preferably at least one eukaryotic expression cassette encoding at least one polypeptide comprising five or more neoantigens.

The live attenuated Salmonella strain, more particularly the Salmonella typhi Ty21a strain, according to the present invention stably carries a recombinant DNA molecule comprising at least one eukaryotic expression cassette encoding at least one tumor antigen, stroma antigen and/or checkpoint inhibitor antigen. The term “ Salmonella typhi Ty21a strain” as used herein refers to an attenuated strain of Salmonella, more specifically of Salmonella typhi, wherein the attenuated strain is Ty21a and is used synonymously with “attenuated strain Salmonella typhi Ty21a” herein.

According to the invention, the Salmonella typhi Ty21a strain functions as the bacterial carrier of the DNA molecule comprising at least one eukaryotic expression cassette encoding at least one tumor antigen, stroma antigen and/or checkpoint inhibitor antigen for the delivery of said DNA molecule into a target cell. Thus, the DNA molecule is a recombinant DNA molecule. Preferably the DNA molecule is a plasmid comprising at least one eukaryotic expression cassette encoding at least one tumor antigen, stroma antigen and/or checkpoint inhibitor antigen. Such a bacterial carrier or delivery vector comprising the DNA molecule comprising at least one eukaryotic expression cassette encoding at least one tumor antigen, stroma antigen and/or checkpoint inhibitor antigen may also be referred to as DNA vaccine. Thus the invention further relates to a DNA vaccine comprising a Salmonella typhi Ty21a strain comprising a DNA molecule comprising at least one eukaryotic expression cassette encoding at least one tumor antigen, stroma antigen and/or checkpoint inhibitor antigen for the use in the treatment of cancer in a human subject following treatment with an antibiotic, wherein the DNA vaccine comprising the Salmonella typhi Ty21a strain is to be administered orally.

Genetic immunization might be advantageous over conventional vaccination. The target DNA can be detected for a considerable period of time thus acting as a depot for the antigen. Sequence motifs in some plasmids, like CpG islands, are immunostimulatory and can function as adjuvants furthered by the immunostimulation due to LPS and other bacterial components.

In the context of the present invention, the term “vaccine” refers to an agent which is able to induce an immune response in a subject upon administration. A vaccine can preferably prevent, ameliorate or treat a disease. In the context of the present invention the vaccine is an oral vaccine. The Salmonella typhi Ty21a strain comprising a DNA molecule comprising at least one eukaryotic expression cassette encoding at least one tumor antigen, stroma antigen and/or checkpoint inhibitor antigen according to the invention may be abbreviated to “ Salmonella typhi Ty21a strain encoding at least one antigen” or “cancer vaccine”. In a preferred embodiment the Salmonella typhi Ty21a strain comprising a DNA molecule comprising at least one eukaryotic expression cassette encodes at least one tumor antigen and/or stroma antigen, more preferably a tumor antigen or a stroma antigen.

Live attenuated Salmonella vectors produce their own immunomodulatory factors such as lipopolysaccharides (LPS) in situ which may constitute an advantage over other forms of administration such as microencapsulation. Moreover, the mucosal vaccine according to the present invention has an intra-lymphatic mode of action, which proved to be beneficial. After ingestion of the attenuated vaccine according to the present invention, macrophages and other cells in Peyer’s patches of the gut are invaded by the modified bacteria. The bacteria are taken up by these phagocytic cells. Due to their attenuating mutations, bacteria of the S. typhi Ty21 strain are not able to persist in these phagocytic cells and die. The recombinant DNA molecules are released and subsequently transferred into the cytosol of the phagocytic immune cells, either via a specific transport system or by endosomal leakage. Finally, the recombinant DNA molecules enter the nucleus, where they are transcribed, leading to massive expression of the at least one tumor antigen, stroma antigen and/or checkpoint inhibitor antigen in the cytosol of the phagocytic cells. The infected cells undergo apoptosis, loaded with the at least one tumor antigen, stroma antigen and/or checkpoint inhibitor antigen, and are taken up and processed by the gut’s immune system. The danger signals of the bacterial infection serve as a strong adjuvant in this process, leading to a strong target antigen specific CD8+T-cell and antibody response at the level of both systemic and mucosal compartments. The immune response peaks around ten days after vaccination. The lack of anti-carrier response allows boosting with the same vaccine several times.

In the context of the present invention, the term “attenuated” refers to a bacterial strain of reduced virulence due to an attenuating mutation compared to the parental bacterial strain, not harboring the attenuating mutation. Attenuated bacterial strains have preferably lost their virulence but retain their ability to induce protective immunity. Attenuation can be accomplished by deletion of various genes, including virulence, regulatory, and metabolic genes. Attenuated bacteria may be found naturally or they may be produced artificially in the laboratory, for example by adaptation to a new medium or cell culture conditions or they may be produced by recombinant DNA technology. Administration of about 10¹¹ CFU of the attenuated strain of Salmonella according to the present invention preferably causes Salmonellosis in less than 5%, more preferably less than 1%, most preferably less than 1‰ of subjects. The strain Ty21a according to the invention is an attenuated strain of Salmonella typhi.

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. In one embodiment the term “comprising” may be individually replaced by the term “consisting of”. The term “a” as used herein may include the plural and hence includes, but is not limited to “one”.

The term “antigen” as used herein refers to any protein or peptide suitable for inducing an immune response. However, in the context of the Salmonella typhi Ty21a strain according to the invention the term “antigen” relates to a tumor antigen, a tumor stroma antigen or a checkpoint inhibitor antigen, wherein the tumor antigen may be a tumor-specific antigen (including a neoantigen) or a tumor-associated antigen. The term “tumor antigen” as used herein refers to an antigen that is exclusively expressed or overexpressed in a tumor, preferably a solid tumor. Thus in certain embodiments the Salmonella typhi Ty21a strain comprises a DNA molecule comprising at least one eukaryotic expression cassette encoding at least one neoantigen. In a preferred embodiment the at least one neoantigen is expressed as at least one polypeptide comprising five or more neoantigens.

In one embodiment the Salmonella typhi Ty21a strain comprises a DNA molecule comprising at least one eukaryotic expression cassette encoding at least one antigen selected from the group consisting of human Wilms’ Tumor protein (WT1), human mesothelin (MSLN), human CEA, CMV pp65, human PD-L1, human VEGFR-2 and human fibroblast activation protein (FAP), preferably human VEGFR-2. The Salmonella typhi Ty21a strain of the present invention may also comprise a DNA molecule comprising at least one eukaryotic expression cassette encoding at least one polypeptide comprising one, two, three, four, five or more antigens selected from the group consisting of tumor antigen, stroma antigen and checkpoint inhibitor antigen, preferably comprising human VEGFR-2.

In another embodiment or in addition the Salmonella typhi Ty21a strain comprises a DNA molecule comprising at least one eukaryotic expression cassette encoding at least one polypeptide comprising five or more neoantigens. In certain embodiments the five or more neoantigens are tumor specific antigens identified in the solid tumor of said subject.

Examples for tumor antigens, particularly human tumor antigens are, without being limited thereto, human Wilms' tumor protein (WT1), human mesothelin (MSLN), human carcinoembryonales antigen (CEA), Human Epidermal Growth Factor Receptor 2 (HER2), epidermal growth factor receptor (EGFR), folate-binding protein (FBP), ganglioside GD2, ganglioside GD3, human programmed death-ligand 1 (PD-L1), vascular endothelial growth factor receptor 2 (VEGFR-2), human fibroblast activation protein (FAP), melanoma antigen A1 (MAGE-A1), prostate stem cell antigen (PSCA), prostate-specific membrane antigen (PSMA), mucin-1 (MUC1), glypican-3 (GPC3), epithelial cell adhesion molecule (EpCAM), B-cell maturation antigen (BCMA) and tyrosine-protein kinase transmembrane receptor (ROR1) and anti-cytomegalovirus pp65 (CMV pp65), wherein the tumor antigen may for example be expressed by the solid tumors as listed in Table 1:

| | |
|---|---|
| Tumor antigen | Solid tumor |
| CEA | Colorectal cancer, breast cancer, hepatocellular cancer |
| EGFR | Glioma (including glioblastoma); lung cancer, particularly non-small cell lung cancer |
| FBP | Ovarian cancer |
| GD2 | Neuroblastoma, glioblastoma |
| GD3 | Glioblastoma, melanoma |
| HER2 | Carcinomas such as glioblastoma, glioma, sarcoma, head and neck squamous cell carcinoma, breast cancer, ovarian cancer, gastric cancer, lung cancer, pancreatic cancer |
| MAGE-A1 | Lung cancer, melanoma, head and neck cancer |
| MSLN | Metastatic cancer, mesothelioma, pancreatic cancer, breast cancer, lung cancer |
| PSCA | Prostate cancer |
| PSMA | Prostate cancer |
| MUC1 | Carcinomas such as glioblastoma, glioma, breast cancer, gastric cancer, lung cancer, pancreatic cancer, colorectal cancer, hepatocellular cancer |
| GPC3 | Lung cancer, particularly non-small cell lung cancer, hepatocellular cancer |
| WT1 | Glioma (including glioblastoma), ovarian cancer, lung cancer |
| EpCAM | Colorectal cancer, renal cancer, prostate cancer |
| BCMA | Breast cancer |
| ROR1 | Ovarian cancer |

In a particular example, the Salmonella typhi Ty21a strain of the present invention comprises a DNA molecule comprising at least one eukaryotic expression cassette encoding at least one antigen selected from the group consisting of WT1, MSLN, CEA, CMVpp65, PD-L1, VEGFR-2 and FAP. In a further example, the Salmonella typhi Ty21a strain of the present invention may comprise a DNA molecule comprising at least one eukaryotic expression cassette encoding at least one polypeptide comprising one, two, three, four, five or more antigens. In a particular example, the Salmonella typhi Ty21a strain comprises a DNA molecule comprising at least one eukaryotic expression cassette encoding at least one polypeptide comprising five or more neoantigens.

In particular embodiments human VEGFR-2 comprises the amino acid sequence of SEQ ID NO: 1 or an amino acid sequence that has at least 80% sequence identity with the amino acid sequence of SEQ ID NO: 1. In particular embodiments human Wilms' Tumor Protein (WT1) comprises the amino acid sequence of SEQ ID NO: 3 or an amino acid sequence that has at least 80% sequence identity with the amino acid sequence of SEQ ID NO: 3. In particular embodiments human Mesothelin (MSLN) comprises the amino acid sequence of SEQ ID NO: 4 or an amino acid sequence that has at least 80% sequence identity with the amino acid sequence of SEQ ID NO: 4. In particular embodiments human CEA comprises the amino acid sequence of SEQ ID NO: 5 or an amino acid sequence that has at least 80% sequence identity with the amino acid sequence of SEQ ID NO: 5. In particular embodiments CMV pp65 comprises the amino acid sequence of SEQ ID NO: 6, 7 or 8 or an amino acid sequence that has at least 80% sequence identity with the amino acid sequence of SEQ ID NO: 6, 7 or 8. In particular embodiments human PD-L1 comprises the amino acid sequence of SEQ ID NO: 9 or 10, or an amino acid sequence that has at least 80% sequence identity with the amino acid sequence of SEQ ID NO: 9, 10 or 11.

Preferably VEGFR-2 has the amino acid sequence of SEQ ID NO: 1, WT1 has the amino acid sequence of SEQ ID NO: 3, MSLN has the amino acid sequence of SEQ ID NO: 4, CEA has the amino acid sequence of SEQ ID NO: 5, CMV pp65 has the amino acid sequence of SEQ ID NO: 6, 7 or 8 and/or PD-L1 has the amino acid sequence of SEQ ID NO: 9, 10 or 11.

VEGFR-2, also known as kinase-insert-domain-containing receptor (KDR), appears to mediate almost all of the known cellular responses to VEGF. For example, the role of VEGF in angiogenesis appears to be mediated through the interaction of this protein with VEGFR-2. VEGFR-2 is a 1356 amino acid long, 200-230 kDa molecular weight high-affinity receptor for VEGF, as well as for VEGF-C and VEGF-D. Identified in humans through the screening of endothelial cDNA for tyrosine kinase receptors, VEGFR-2 shares 85% sequence identity with the previously discovered mouse fetal liver kinase 1 (Flk-1). VEGFR-2 is normally expressed in endothelial and hematopoietic precursors, as well as in endothelial cells, nascent hematopoietic stem cells and the umbilical cord stroma. However, in quiescent adult vasculature, VEGFR-2 mRNA appears to be down regulated.

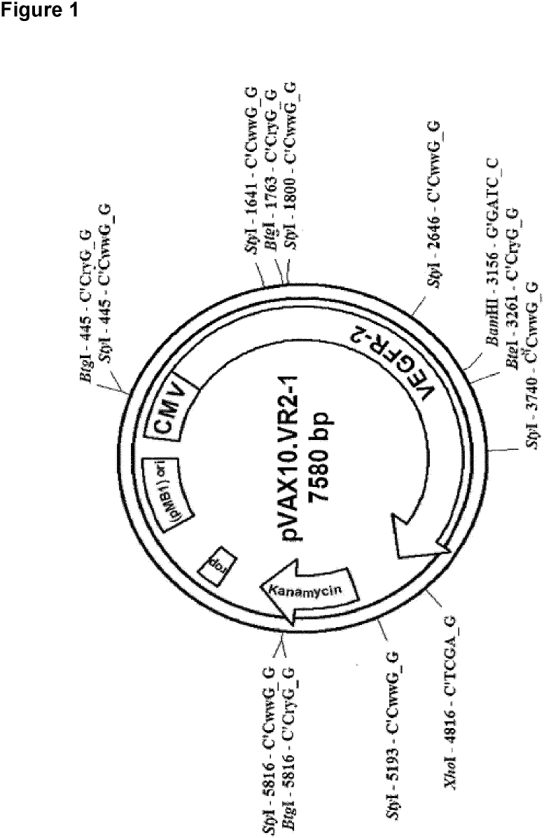

The extracellular domain of VEGFR-2 contains 18 potential N-linked glycosylation sites. VEGFR-2 is initially synthesized as a 150 kDa protein and rapidly glycosylated to a 200 kDa intermediate form, and then further glycosylated at a slower rate to a mature 230 kDa protein which is expressed on the cell surface. In one embodiment the at least one tumor antigen, tumor stroma antigen and/or checkpoint inhibitor antigen comprises or is the extracellular domain of VEGFR-2. The Salmonella typhi Ty21a strain comprising a DNA molecule comprising at least one eukaryotic expression cassette encoding VEGFR-2 is also referred to as VXM01. More specifically VXM01 comprises the plasmid as shown in FIG. 1.

VEGF receptors have long been assumed to be restricted to the vasculature of malignancies, i.e. to the tumor stroma. Recent expression analyses, however, revealed the expression of vascular endothelial growth factor receptors, in particular VEGFR-2, on tumor cells themselves. Tumor-specific VEGF receptor expression was observed on cancer cells of various origins. This indicates that VEGF might have additional effects on tumorigenesis besides promoting neovascularization. Examples for cancers characterized by VEGFR-2 expressing cancer cells are, without being limited thereto, glioblastoma, carcinoid cancer, kidney cancer, particularly renal cell carcinoma, thyroid cancer, lung cancer, particularly Non-Small Cell Lung Cancer (NSCLC), breast cancer, ovarian cancer, prostate cancer, gastrointestinal cancer, particularly colorectal cancer, more particularly colon cancer, and skin cancer, particularly melanoma.

One particularly promising indication for VEGFR-2 targeting immunotherapy is glioblastoma. Glioblastoma shows extremely high tumor vascularization. Moreover, VEGFR-2 may be targeted on both the tumor vasculature and the tumor cells. About 20% to 50% of glioblastoma patients show tumor-specific VEGFR-2 expression, which is particularly observed at the invasion front. Furthermore, VEGFR-2 expression was observed in glioma-like stem cells. So far, the treatment options for glioblastoma remain unsatisfactory. For example, the monoclonal antibody avastin targeting VEGF only showed benefits in progression free survival, but not in overall survival.

It is therefore also encompassed by the present invention that the human subject has been determined to have a cancer characterized by VEGFR-2 expressing cancer cells or to have at least one VEGFR-2 expressing cancer cell. In a first step, the subject’s tumor-specific VEGFR-2 expression, e.g. the tumor-specific expression of VEGFR-2, may be assessed on mRNA or protein level, preferably in vitro. For that purpose, tumor tissue samples (e.g., a biopsy) may for example either be stained by immunohistochemistry staining or they may undergo in situ hybridization. Methods for the assessment of tumor-specific antigen expression are well known in the art. The same applies to determining whether the human subject has a cancer characterized by tumor antigen expressing cancer cells, particularly human WT1, human MSLN, human CEA, CMV pp65, human PD-L1, and human FAP expressing cancer cells or whether the human subject has at least one tumor antigen expressing cancer cell, particularly human WT1, human MSLN, human CEA, CMV pp65, human PD-L1, and human FAP expressing cancer cell. The person skilled in the art would immediately understand that a Salmonella typhi Ty21a strain comprises a DNA molecule comprising at least one eukaryotic expression cassette encoding human WT1, human MSLN, human CEA, CMV pp65, human PD-L1 and/or FAP may be used for treating cancer in a human subject that has been determined to have a cancer characterized by WT1, MSLN, CEA, CMV pp65, PD-L1, and/or FAP expressing cancer cells or to have at least one WT1, MSLN, CEA, CMV pp65, PD-L1, and/or FAP expressing cancer cell, respectively.

Mesothelin is a 40-kDa cell surface glycoprotein present on normal mesothelial cells and overexpressed in several human tumors, including mesothelioma and ovarian and pancreatic adenocarcinoma. The mesothelin gene encodes a precursor protein of 71-kDa that is processed to yield a 31-kDa shed protein named megakaryocyte-potentiating factor (MPF) and the 40-kDa cell bound fragment mesothelin. Mesothelin was shown to exhibit megakaryocyte-colony-forming activity in the presence of interleukin-3. Mesothelin is a tumor differentiation antigen present at low levels on a restricted set of normal adult tissues, such as mesothelium, but aberrantly overexpressed in a wide variety of human tumors including mesotheliomas, ovarian and pancreatic cancers, squamous cell carcinomas of the cervix, head and neck, vulva, lung and esophagus, lung adenocarcinomas, endometrial carcinomas, biphasic synovial sarcomas, desmoplastic small round cell tumors and gastric adenocarcinomas. The normal biological function of Mesothelin is unknown. Studies in mesothelin knock-out mice revealed no detectable phenotype, and both male and female mice produced healthy off-spring. Studies in pancreatic cancer suggest that mesothelin plays a role in tumorigenesis by increasing cellular proliferation, migration, and S-phase cell populations. Furthermore, there is evidence that mesothelin is an immunogenic protein. Due to its expression profile, its oncogenic functions and its immunogenic potential, the tumor antigen mesothelin is a promising candidate for the development of cancer vaccines.

Wilms' tumor gene 1 (WT1) encodes a zinc finger transcription factor involved in cell proliferation and differentiation. The WT1 protein contains four zinc finger motifs at the C-terminus and a proline/glutamine-rich DNA-binding domain at the N-terminus. Multiple transcript variants, resulting from alternative splicing at two coding exons, have been well characterized. WT1 plays an essential role in the development of the urogenital system and is involved in cell proliferation and differentiation. The WT1 gene was isolated as the gene responsible for a childhood renal neoplasm, Wilms' tumor. It is highly expressed in a wide variety of malignancies including several types of hematological malignancies and various solid tumors. In contrast, normal tissue expression of WT1 in adults is restricted to gonads, uterus, kidney, mesothelium and progenitor cells in various types of tissues. WT-1 negatively affects differentiation and promotes proliferation of progenitor cells. Furthermore, overexpressed WT1 is immunogenic; WT1 specific T-cells as well as IgG anti-WT1 antibodies have been observed in cancer patients. Due to its expression profile, its oncogenic functions and its immunogenic potential, the tumor antigen WT1 is a promising candidate for the development of cancer vaccines. In particular embodiments, WT1 is truncated. In particular embodiments, the zinc finger domain of WT1 is deleted. In particular embodiments, the truncated WT1 has the amino acid sequence of SEQ ID NO: 3.

The zinc finger domain at the C-terminus of WT1 comprises four zinc finger motifs. Truncated WT1 of the amino acid sequence of SEQ ID NO: 3 represents amino acids 1 to 371 of UniProt ref P19544-7. Deletion of the zinc finger domain minimizes the risk of immunological cross reactivity with other zinc finger containing transcription factors. Furthermore, truncated WT1 lacking the zinc finger domain has greater immunogenic potential than full-length WT1. In addition, deletion of the zinc finger motifs, which are essential for DNA binding, abrogates the oncogenic potential of WT1, thus minimizing the risk of oncogenesis.

The tegument protein CMV pp65 is a major immunodominant protein of human cytomegalovirus (CMV). The biologic function of CMV pp65 is unclear, but it is believed to be involved in cell cycle regulation. CMV pp65 is a nucleotropic protein exhibiting protein kinase activity, which is able to bind polo-like kinase 1 (PLK-1). Human CMV pp65 is expressed in more than 90% of glioblastoma specimens but not in surrounding normal brain. This viral protein is thus a promising candidate as tumor-specific target for the development novel of cancer immunotherapies.

The CMV pp65 protein contains two bipartite nuclear localization signals (NLSs) at amino acids 415 to 438 and amino acids 537 to 561 near the carboxy terminus and a phosphate binding site related to its kinase activity at lysine-436. Mutating the lysine at position 436 to asparagine and deletion of amino acids 537 to 561 results in a protein without kinase activity and markedly reduced nuclear localization. This mutant protein exhibits unaltered immunogenicity.

In particular embodiments, the CMV pp65 has the amino acid sequence of SEQ ID NO: 6. SEQ ID NO: 6 representing the amino acid sequence of wild type human CMV pp65. In particular other embodiments, the CMV pp65 has the amino acid sequence of SEQ ID NO: 7. SEQ ID NO: 7 represents the amino acid sequence of human CMV pp65, which harbors the mutation K436N relative to the wild type human CMV pp65 having the amino acid sequence of SEQ ID NO: 6. In particular other embodiments, the CMV pp65 has the amino acid sequence of SEQ ID NO: 8. SEQ ID NO: 8 represents the amino acid sequence of a truncated version of CMV pp65 having the amino acid sequence of SEQ ID NO: 7, which lacks the second, more C-terminal NLS (nuclear localization sequence) (i.e. amino acids 537 to 561 of CMV pp65 of SEQ ID NO: 7).

Carcinoembryonic antigen (CEA) (also known as CEACAM5 and CD66e) is a member of a family of highly related glycosyl phosphatidyl inositol (GPI) cell surface anchored glycoproteins involved in cell adhesion. CEA is normally produced in gastrointestinal tissue during fetal development; protein expression ends before birth. Therefore CEA is usually present only at very low levels in the blood of healthy adults. However, the serum levels are raised in some types of cancer, in particular colorectal carcinoma, thus serving as tumor marker. CEA levels may also be raised in gastric carcinoma, pancreatic carcinoma, lung carcinoma, breast carcinoma, and medullary thyroid carcinoma, as well as some non-neoplastic conditions like ulcerative colitis, pancreatitis, cirrhosis, COPD, Crohn’s disease and hypothyroidism.

Programmed cell death 1 (PD-1) is expressed on the surface of T-cells and transmits inhibitory signals that maintain T-cell functional silence against cognate antigens. Its ligand PD-L1 is normally expressed on antigen-presenting cells, placental cells and nonhematopoietic cells in inflammatory microenvironments. PD-L1 has been reported to be expressed on immunosuppressive myeloid-derived suppressor cells (MDSC). In addition, PD-L1 is extensively expressed on the surface of various types of cancer cells, which use the PD-⅟PD-L1 signaling axis to escape the host immune system. Expression of PD-L1 by cancer cells was shown to correlate with disease stage and poor patient prognosis.

In particular embodiments, PD-L1 is selected from the group consisting of full length PD-L1 and a truncated PD-L1 comprising the extracellular domain of PD-L1. A truncated PD-L1 may comprise an amino acid sequence of amino acids 19 to 238 of SEQ ID NO: 11, the amino acid sequence of SEQ ID NO: 11, the amino acid sequence of SEQ ID NO: 10 or may comprise an amino acid sequence that shares at least 80% sequence identity with amino acids 19 to 238 of SEQ ID NO: 11, with SEQ ID NO: 11 or with SEQ ID NO: 10. In particular embodiments the PD-L1 is selected from the group consisting of PD-L1 having the amino acid sequence of SEQ ID NO: 9 and a protein that shares at least 80% sequence identity therewith. In particular other embodiments PD-L1 is selected from the group consisting of PD-L1 having the amino acid sequence of SEQ ID NO: 10 and a protein that shares at least 80% sequence identity therewith. In particular other embodiments PD-L1 is selected from the group consisting of PD-L1 having the amino acid sequence of SEQ ID NO: 11 and a protein that shares at least 80% sequence identity therewith. In particular other embodiments PD-L1 is selected from the group consisting of PD-L1 having the amino acid sequence of amino acids 19 to 238 of SEQ ID NO: 11 and a protein that shares at least 80% sequence identity therewith. Particularly, PD-L1 has the amino acid sequence of SEQ ID NO: 9, SEQ ID NO: 10 or SEQ ID NO: 11. Preferably PD-L1 comprises the amino acid sequence of amino acids 19 to 238 of SEQ ID NO: 11. In one embodiment PD-L1 comprises at least the extracellular domain with or without the signaling peptide.

As used herein, the term “about” or “approximately” means within 80% to 120%, alternatively within 90% to 110%, including within 95% to 105% of a given value or range.

In the context of the present invention, the term “protein that shares at least 80% sequence identity with the amino acid sequence of SEQ ID NO: X” refers to a protein that has an amino acid sequence with more than 80% amino acid identity when aligned with the amino acid sequence provided. The protein may be of natural origin, e.g. a mutant version of a wild-type protein, e.g. a mutant version of a wild type VEGFR-2 protein, or a homolog of a different species, or an engineered protein, e.g. an engineered VEGFR-2 protein. Methods for designing and constructing derivatives of a given protein are well known to anyone of ordinary skill in the art.

The protein that shares at least 80% sequence identity with a given amino acid sequence may contain one or more mutations comprising an addition, a deletion and/or a substitution of one or more amino acids in comparison to the reference amino acid sequence. According to the teaching of the present invention, said deleted, added and/or substituted amino acids may be consecutive amino acids or may be interspersed over the length of the amino acid sequence of the protein that shares at least 80% sequence identity with a given reference protein. According to the teaching of the present invention, any number of amino acids may be added, deleted, and/or substitutes, as long as the amino acid sequence identity with the reference amino acid sequence is at least 80% and the mutated protein is immunogenic. Preferably, the immunogenicity of the protein which shares at least 80% sequence identity with the reference amino acid sequence is reduced by less than 50%, less than 40%, less than 30%, less than 20%, less than 10%, less than 5% or less than 1% compared to the reference amino acid sequence, as measured by ELISA. Methods for designing and constructing protein homologues and for testing such homologues for their immunogenic potential are well known to anyone of ordinary skill in the art. In particular embodiments, the sequence identity with the reference amino acid is at least 85%, at least 90%, at least 95% and most particularly at least 99%. Methods and algorithms for determining sequence identity including the comparison of a parental protein and its derivative having deletions, additions and/or substitutions relative to a parental sequence, are well known to the practitioner of ordinary skill in the art. On the DNA level, the nucleic acid sequences encoding the protein that shares at least 80% sequence identity with the reference amino acid sequence may differ to a larger extent due to the degeneracy of the genetic code.

A tumor antigen is an antigen that is exclusively expressed or overexpressed in a tumor, preferably a solid tumor. Thus, a tumor antigen may be a tumor-specific antigen, or a tumor-associated antigen. Tumor-specific antigens encompass neoantigens. Thus, in certain embodiments the Salmonella typhi Ty21a strain comprises a DNA molecule comprising at least one eukaryotic expression cassette encoding at least one neoantigen. In a preferred embodiment the at least one neoantigen is/are expressed as at least one polypeptide comprising five or more neoantigens. Preferably the five or more neoantigens are tumor specific antigens identified in the solid tumor of said subject.

The term “neoantigens” as used herein relates to peptides that are generated from somatically mutated genes expressed only in cancer cells, but not in normal tissue of the same patient. Genes and chromosomes can mutate in either somatic or germinal tissue. Opposite to germline mutations, somatic mutations are not transmitted to progeny. Thus, the somatic mutations in the gene have been acquired in the cancer cells and during cancer development. Typically the mutation is a tumor-specific point mutation generating a neoepitope also referred to as a mutational epitope or point-mutated peptide. They are highly immunogenic because they are not present in normal tissues and hence bypass central thymic tolerance. Neoantigens comprise, preferably consist of, the neoepitope presented as peptide by MHC I or MHC II. The mutation may also be a frameshift mutation resulting in a frameshift peptide (FSP) antigen. FSP neoantigens, although caused by insertion or deletion of single nucleotides, encompass long antigenic amino acid stretches, which can contain multiple immunologically relevant neoepitopes. In specific embodiments, the term “neoantigen” further includes T cell epitopes associated with peptide processing (TEIPP). TEIPPs are derived from ubiquitously expressed non-mutated “self” proteins that are not loaded into MHC I in healthy cells. In immune-escaping cancers antigen-processing components, like the transporter associated with antigen processing (TAP) are often downregulated. Thus, only in cells with defects in the antigen-processing machinery, such as in the absence of TAP due to mutations or epigenetic silencing, TEIPPs may be presented on the surface of cancer cells (Marjit et al., Journal of Experimental Medicine, 2018, 215(9): 2325).

During cancer progression, mutations accumulating in the cancer genome can affect protein-coding genes and result in altered protein sequences. Mutated proteins are proteolytically cleaved into short peptides and presented on the tumor cell surface by MHC (human leucocyte antigen (HLA) in humans). These somatically mutated genes, i.e., neoantigens, which are presented in the malignant cells but not in the normal cells can be recognized as foreign by tumor-infiltrating lymphocytes (TILs). Thus, the term neoantigen refers to a peptide comprising, preferably consisting of, the peptide containing the somatic mutation that is presented by MHC I or II. Neoantigens presented by MHC I may also be referred to as CD8 T cell antigens. Neoantigens presented by MHC II may also be referred to as CD4 T cell antigens (or T helper antigens). As neoantigens can be recognized as foreign by TILs they are capable of eliciting potent tumor specific immune responses. Neoantigens released after tumor cell death initiate a number of processes that ultimately lead to T cells that recognize cancer cells through the interaction of distinct T-cell receptors (TCR) with specific neoantigen-MHC complexes.

The term “at least one polypeptide comprising five or more neoantigens” as used herein refers to one polypeptide or more than one polypeptide comprising together 5 or more neoantigens. Whether the five or more neoantigens are part of the same or different polypeptides is not relevant. The five or more neoantigens may therefore be expressed as one polypeptide or as more than one polypeptide. Preferably the neoantigens comprised within the at least one or more polypeptide(s) are 10 or more, 20 or more, 30 or more, 50 or more, or more than 50 neoantigens. In the context of the Salmonella typhi Ty21a strain as used herein, the insert encoding the at least one polypeptide may comprise up to 300 neoantigens, preferably up to 200 neoantigens. Antigens presented as peptides on MHC class I or II (in humans HLA) are typically from 11 to 30 amino acids long for MHC II (CD4 antigens) and from 8 to 10 amino acids for MHC I (CD8 antigens). Thus, the five or more neoantigens may preferably comprise CD8 T-cells antigens or CD8 and CD4 T-cell antigens. Further, the preferred ranges for neoantigens to be contained within the at least one polypeptide may be 5 to 300, 10 to 300, 20 to 300, 30 to 300, 50 to 300, or more than 50 to 300 neoantigens. More preferred ranges for neoantigens to be contained within the at least one polypeptide may be 5 to 200, 10 to 200, 20 to 200, 30 to 200, 50 to 200, or more than 50 to 200 neoantigens. Each polypeptide comprising fused neoantigens may be proteolytically cleaved into the neoantigens inside antigen presenting cells and presented via HLA to elicit a T-cell response.

According to the invention, the five or more neoantigens may comprise CD8 T cell antigens and/or CD4 T cell antigens. Preferably, the five or more neoantigens comprise CD8 T cell antigens and CD4 T cells antigens.

It is hypothesized that vaccination with neoantigens can both expand pre-existing neoantigen-specific T-cell populations and induce a broader repertoire of new T-cell specificities in cancer patients.

A neoantigen is typically a peptide having 8 to 30 amino acids, preferably 8 to 20, more preferably 8 to 12 amino acids.

For a neoantigen cancer vaccine it is beneficial if the vaccine targets multiple neoantigens, thus reducing the risk of immune-evasion due to loss of expression of subsets of neoantigens. It is also encompassed by the invention that the human subject is treated sequentially with another Salmonella typhi Ty21a strain comprising a DNA molecule comprising at least one eukaryotic expression cassette encoding at least one polypeptide comprising five or more neoantigens, comprising targeting new neoantigens or a new subset of neoantigens selected during tumor progression.

Advantage of the attenuated strain of Salmonella typhi Ty21a, also referred to as “ Salmonella typhi Ty21a”, as carrier for the at least one polypeptide comprising five or more neoantigens are the a) established quality control assay, b) the individual differences of the plasmid only in the insert encoding the one or more neoantigens, c) no need for expansion and d) no requirements with regard to sterility testing due to oral administration. Furthermore, expression plasmids suitable for transformation as well as the Salmonella typhi Ty21a strain as carrier allow a high number (up to 300) of epitopes (neoantigens). The neoantigens may be inserted into the plasmid as a string of beads (expressed as one or more polypeptides), optionally separated by a linker. The linker may be, without being limited thereto, a GS linker, a 2A cleavage site, or an IRES sequence. Due to the fast generation and only limited need for quality control, the time for generating the Salmonella typhi Ty21a strain comprising a DNA molecule comprising at least one eukaryotic expression cassette encoding at least one polypeptide comprising five or more neoantigens is short and can for example be achieved within 15 days, preferably within 14 days or less after identification of the neoantigens. Overnight fermentation is sufficient and no upscaling is required due to high yield of bacteria with a net yield in the range of 10¹¹ colony forming units (CFU) in a 1 L culture. This allows for the short manufacturing time, as well as the low manufacturing costs. Furthermore, each batch is sufficient for years of treatment and the drug product was shown to be stable for at least three years. Thus, no batch variation will occur, since one batch lasts for the entire treatment of the human subject having the solid tumor.

A method for generating a Salmonella typhi Ty21a strain comprising a DNA molecule comprising at least one eukaryotic expression cassette encoding at least one polypeptide comprising five or more neoantigens for an individual subject with a solid tumor comprises (a) providing a tumor cell sample and a control sample from said subject; (b) identifying five or more neoantigens present in the tumor cell sample that are not present in the control sample; (c) selecting five or more neoantigens; (d) synthesizing a cDNA encoding the at least one polypeptide comprising five or more neoantigens; (e) cloning the cDNA into the at least one eukaryotic expression cassette; (f) transforming a Salmonella typhi Ty21a recipient strain with the DNA molecule comprising at least one eukaryotic expression cassette encoding at least one polypeptide comprising five or more neoantigens; (g) fermenting the strain obtained in step (f) and diluting to target concentration based on CFU; and (h) analyzing said transformed Salmonella typhi Ty21a strain comprising sequencing the cDNA encoding the at least one polypeptide comprising five or more neoantigens. The control sample may be any sample of normal tissue or blood from the subject to be treated. The term “normal tissue” refers to non-cancerous tissue, preferably from the same tissue, i.e., origin. Preferably the control sample is a blood sample. The blood sample may further be used for HLA typing of the patient. The tumor cell sample may be a tumor biopsy.

Methods for detecting (all) coding mutations within tumors and reliably predicting or determining those mutated peptides with high-affinity binding of autologous human leukocyte antigen (HLA) molecules are known in the art. For example whole-exome sequencing (WES) of matched tumor and normal cell DNA from individual patients can be performed. Identified somatic mutations are then orthogonally validated and assessed for expression of mutated alleles by RNA sequencing of the tumor. Peptides are then selected that are predicted to likely bind to autologous HLA-A or HLA-B proteins of the patient. This may be confirmed, e.g., by ex vivo interferon ʏ enzyme-linked immunospot (ELISpot). Alternatively, HLA-peptide ligands can be isolated from cell media and identification can be conducted by LC-MS/MS analysis.

A polypeptide may comprise several neoantigens fused to each other, preferably 5 or more, 10 or more, 20 or more, 30 or more, or 50 or more neoantigens. In a typical plasmid used to transfect the Salmonella typhi Ty21a strain, such as pVAX1™ expression plasmid (Invitrogen, San Diego, California) or pVAX10 derived thereof, up to about 300 neoantigens may be expressed. The polypeptide may therefore comprise about 5 to 300, 10 to 300, 20 to 300, 30 to 300 or 50 to 300 neoantigens, preferably 10 to 200, 20 to 200, 30 to 300, or 50 to 200 neoantigens. The polypeptide is cleaved intracellularly into peptide and presented on MHC I or MHC II molecules, depending on the type of neoantigen. The individual neoantigens may be separated by a linker, such as a GS linker, specifically designed linkers or a 2A cleavage site. The DNA molecule encoding the neoantigen(s) may also be separated by an IRES sequence, resulting in separate polypeptides.

The Salmonella typhi Ty21a strain comprising a DNA molecule comprising at least one eukaryotic expression cassette encoding at least one polypeptide comprising five or more neoantigens may further comprising a DNA molecule comprising at least one eukaryotic expression cassette encoding at least one polypeptide comprising at least one tumor antigen that is not a neoantigen and/or tumor stroma antigen, wherein said at least one tumor antigen that is not a neoantigen is expressed in the solid tumor of the patient to be treated. The term “at least one tumor antigen that is not a neoantigen and/or tumor stroma antigen” as used herein means at least one tumor antigen and/or stroma antigen, wherein the tumor antigen is not a neoantigen. The at least one polypeptide comprising at least one tumor antigen that is not a neoantigen and/or tumor stroma antigen (a) may be encoded by the same DNA molecule comprising the at least one eukaryotic expression cassette encoding the at least one polypeptide comprising five or more neoantigens or by a further separate DNA molecule, (b) may be encoded by the at least one eukaryotic expression cassette encoding the at least one polypeptide comprising five or more neoantigens or by a further separate expression cassette; or (c) may be the at least one polypeptide comprising five or more neoantigens or a further separate polypeptide. Thus, the Salmonella typhi Ty21a strain may be transformed with two DNA molecules, the first encoding the five or more neoantigens and the second encoding the at least one tumor antigen that is not a neoantigen and/or tumor stroma antigen. Alternatively, the Salmonella typhi Ty21a strain may be transformed with one DNA molecules, comprising at least one eukaryotic expression cassette encoding at least one polypeptide comprising five or more neoantigens and at least one further eukaryotic expression cassette encoding at least one tumor antigen that is not a neoantigen and/or tumor stroma antigen. Alternatively, the Salmonella typhi Ty21a strain may also be transformed with one DNA molecules, comprising at least one eukaryotic expression cassette encoding at least one polypeptide comprising five or more neoantigens and further comprising at least one tumor antigen that is not a neoantigen and/or tumor stroma antigen. Examples for a tumor antigen that is not a neoantigen in this context are, without being limited thereto, WT1, MSLN, CEA, HER2, EGFR, FBP, GD2, GD3, MAGE-A1, PSCA, PSMA, PD-L1, MUC1, GPC3 and CMV pp65. The tumor antigen may be a tumor specific antigen or a tumor associated antigen. The term “tumor specific antigen” as used herein relates to an antigen expressed in the tumor, but not in normal tissue. The term “tumor associated antigen” as used herein relates to an antigen overexpressed in the tumor compared to normal tissue. The term “tumor stroma antigen” as used herein refers to antigens expressed in the tumor stroma including, without being limited thereto, VEGFR-2 and FAP. The Salmonella typhi Ty21a strain comprising a DNA molecule comprising at least one eukaryotic expression cassette encoding at least one polypeptide comprising five or more neoantigens may also comprise a DNA molecule comprising at least one eukaryotic expression cassette encoding at least one polypeptide comprising a checkpoint inhibitor antigen, wherein said at least one checkpoint inhibitor antigen or its ligand is overexpressed in the solid tumor of the patient to be treated. Thus, the checkpoint inhibitor antigen may also be a tumor antigen, such as PD-L1, which is frequently upregulated on tumor cells. With regard to the expression of the checkpoint inhibitor antigen in the Salmonella typhi Ty21a strain comprising a DNA molecule comprising at least one eukaryotic expression cassette encoding at least one polypeptide comprising five or more neoantigens, the same applies as for the at least one tumor antigen that is not a neoantigen and/or the tumor stroma antigen. An example for a checkpoint inhibitor antigen is PD-1 or PD-L1, other examples are CTLA-4, IDO, GITR, OX40, TIM-3, LAG-3, KIR, CSF1R and CD137. The DNA molecule used in this context is preferably an expression plasmid. PD-L1 may also be regarded as a tumor antigen or tumor-associated antigen.

A DNA molecule comprising at least one eukaryotic expression cassette may also be referred to as a recombinant DNA molecule, i.e. an engineered DNA construct, preferably composed of DNA pieces of different origin. The DNA molecule can be a linear nucleic acid, or preferably, a circular DNA plasmid generated by introducing an open reading frame encoding at least one tumor antigen, stroma antigen and/or checkpoint inhibitor antigen into a eukaryotic expression cassette of a plasmid. A plasmid comprising a eukaryotic expression cassette may also be referred to as eukaryotic expression plasmid.

In the context of the present invention, the term “expression cassette” refers to a nucleic acid unit comprising at least one open reading frame (ORF) under the control of regulatory sequences controlling its expression. Preferably the expression cassette also comprises a transcription termination signal. Expression cassettes can preferably mediate transcription of the included open reading frame encoding at least one tumor antigen, stroma antigen and/or checkpoint inhibitor antigen, in a target cell. Eukaryotic expression cassettes typically comprise a promoter, at least one open reading frame and a transcription termination signal, which allow expression in a eukaryotic target cell.

In particular embodiments, a single dose of the Salmonella typhi Ty21a strain comprises from about 10⁶ to about 10⁹, more particularly from about 10⁶ to about 10⁸, most particularly from about 10⁶ to about 10⁷ colony forming units (CFU).

More particularly, a single dose of the Salmonella typhi Ty21a strain comprises from about 1x10⁶ to about 1×10⁹, more particularly from about 1x10⁶ to about 1×10⁸, most particularly from about 1x10⁶ to about 1×10⁷ colony forming units (CFU).

In this context, the term “about” or “approximately” means within a factor of 3, alternatively within a factor of 2, including within a factor of 1.5 of a given value or range.

Furthermore, the Salmonella typhi Ty21a strain according to the invention is preferably administered two to four times in the first week, preferably 4 times in the first week, followed by single dose boosting administration every 2 to 4 weeks. Preferably the Salmonella typhi Ty21a strain according to the invention is to be administered on day 1 and 7, preferably on day 1, 3, 5 and 7, followed by single dose boosting administrations every 2 to 4 weeks.

In particular embodiments, the treatment comprises a single or multiple administrations of the Salmonella typhi Ty21a strain according to the invention or the pharmaceutical composition or DNA vaccine comprising the Salmonella typhi Ty21a strain according to the invention. The single dose of the administrations may be the same or different, preferably the same and preferably within the ranges as disclosed herein. In one embodiment the treatment comprises a prime vaccination and a boost vaccination. The term “prime vaccination” refers to the initial vaccination priming the immune system, typically comprising two to four single dose vaccinations within the first week. The term “boost vaccination” refers to the following regular and repeated single dose administrations boosting the primed immune system, typically comprising a single dose boosting administration every two to four weeks. Particular, the treatment may comprise two to four prime vaccinations in the first week of treatment followed by single dose boosting administrations every two to four weeks of the Salmonella typhi Ty21a strain encoding at least one tumor antigen, tumor stroma antigen and/or checkpoint inhibitor antigen (including at least one polypeptide comprising five or more neoantigens) or the pharmaceutical composition comprising the Salmonella typhi Ty21a strain according to the present invention.

The Salmonella typhi Ty21a strain encoding at least one tumor antigen, stroma antigen and/or checkpoint inhibitor antigen is for use in the treatment of a cancer of a human subject, preferably of a solid tumor in a human subject, wherein the subject has been or is treated with at least one antibiotic. In a preferred embodiment the at least one tumor antigen, stroma antigen and/or checkpoint inhibitor antigen is for use in the treatment of a cancer of a human subject, preferably of a solid tumor in a human subject following treatment with an antibiotic (i.e., wherein the subject has been treated with at least one antibiotic.

The Salmonella typhi Ty21a strain may be administered after a suitable period of time after completion of the antibiotic treatment. For example the first Salmonella typhi Ty21a strain dose may be administered about 1 day, about 2 days, about 3 days, about 4 days, about 5 days, about 6 days, about 1 week, about 2 weeks, about 3 weeks, about 1 month or about 2 months, preferably about 3 days, 4 days, about 5 days, about 6 days, about 1 week, about 2 weeks, about 3 weeks or about 1 month after completion of the treatment with the antibiotic. In certain embodiments the Salmonella typhi Ty21a strain is to be administered at least 3 days after completion of the treatment with the antibiotic, i.e., 3 days or more than 3 days after completion of the treatment with the antibiotic. In one embodiment the (first dose of the) Salmonella typhi Ty21a strain is administered about 3 days after completion of the treatment with the antibiotic. In another embodiment the (first dose of the) Salmonella typhi Ty21a strain is administered within 1 month of completion of the treatment with the antibiotic. In yet another embodiment the (first dose of the) Salmonella typhi Ty21a strain is administered between about 1 days to one month, preferably 3 days to 1 month, more preferably 3 days to 2 weeks after completion of the treatment with the antibiotic. The term following treatment with an antibiotic” as used herein means after completion of the antibiotic treatment, i.e., after the last dose of the antibiotic. The indicated time or time period or range refers to the first Salmonella typhi Ty21a dose to be administered, while the treatment or administration may continue as long as needed.

Without being bound by theory the antibiotic may impact the intestinal microbiome, which might facilitate uptake of the orally administered Salmonella typhi Ty21a according to the invention. An increased uptake of Salmonella typhi Ty21a is likely to result in an increased expression of the at least one tumor antigen, stroma antigen and/or checkpoint inhibitor antigen in the host cell and hence to an increased presentation of the at least one tumor antigen, stroma antigen and/or checkpoint inhibitor antigen to the immune system, potentially resulting in an increased immune response, particularly T cell mediated immune response, which is particularly important in treating cancer. Thus, any antibiotic suitable for reducing or impacting the intestinal microbiome is suitable in the context of the present invention. The antibiotic may be a single compound or a combination, such as a combination preparation comprising sulfonamides or a β-lactamase inhibitor. Examples for such a β-lactamase inhibitor comprise but are not limited to sulbactam or tazobactam.

Alternatively, the Salmonella typhi Ty21a strain may be administered to a human subject that is or has been treated with an antibiotic. In one embodiment the Salmonella typhi Ty21a strain is to be administered following treatment with an antibiotic, or after completion of the antibiotic treatment. In another embodiment at least the first Salmonella typhi Ty21a strain dose may be administered during treatment with an antibiotic, preferably the two to four administrations in the first week (prime vaccination), may be administered during treatment with an antibiotic and a repeated single dose boosting administration, preferably every 2 to 4 weeks, is to be administered after completion of the treatment with the antibiotic.

Without being bound by theory, in addition to impacting the intestinal microbiome, the concomitant administration of the Salmonella typhi Ty21a strain and the antibiotic in patient No. 0104 may have resulted in killed or inactivated Salmonella typhi Ty21a strain comprising a DNA molecule comprising at least one eukaryotic expression cassette encoding at least one tumor antigen, stroma antigen and/or checkpoint inhibitor antigen, potentially stimulating the immune system and/or resulting in the uptake of the DNA molecule comprising at least one eukaryotic expression cassette encoding at least one tumor antigen, stroma antigen and/or checkpoint inhibitor antigen by host cells via phagocytosis and further expression of the at least one eukaryotic expression cassette encoding at least one tumor antigen, stroma antigen and/or checkpoint inhibitor antigen, resulting in effective priming. However, the progression of the antigen specific immune response as observed in patient No. 0104 in month 6 (following concomitant treatment of the vaccine with the antibiotic for 5 months), accompanied by a reduction of the tumor volume compared to month 3 and even a reduction of the tumor volume compared to base line after 9 months suggests that the vaccine was ineffective during the first 5 months (i.e., during concomitant administration with the antibiotic) and that the boosting every 4 weeks following treatment with the antibiotic was sufficient to induce the antigen specific immune response. The immune response may be even enhanced when the Salmonella typhi Ty21a strain is administered as prime vaccination 2 to 4 times in one week, followed by a single dose boosting administration every 2 to 4 weeks following the treatment with an antibiotic, i.e., when the priming and the boosting is to be administered after the treatment with the antibiotic has been completed, such as when the first dose of the prime vaccination is to be administered about 3 days to about 1 month after the treatment with the antibiotic has been completed followed by the boost vaccination.

The antibiotic may be a broad-spectrum antibiotic, such as the broad-spectrum penicillin antibiotics amoxicillin, ampicillin or piperacillin, or a narrow-spectrum antibiotic, such as a macrolide antibiotic (such as azithromycine, erythromycin, clarithromycin, fidaxomycin or roxithromycin) or vancomycin. The antibiotic may also be a bacteriostatic antibiotic, including but not limited to tetracyclines and sulfonamides, or a bactericidal antibiotic, such as a beta-lactam antibiotic (including penicillin antibiotics).

In certain embodiments the antibiotic is selected from the group consisting of a penicillin (e.g. amoxicillin, ampicillin, piperacillin and flucloxacillin), a cephalosporin, a polymyxin (e.g. colistin), a rifamycin (e.g. rifaximin), a lipiarmycin, a quinolone (e.g. ciprofloxacin), a sulfonamide, a macrolide (e.g. erythromycin), a linocosamide, a tetracycline (e.g., tetracycline), an aminoglycoside (e.g. paromomycin and neomycin), a cyclic lipopeptide (e.g. daptomycin), a glycylcycline (e.g. tigecycline), an oxozolidinone (e.g. linezolid), a nitrodimazole (e.g. metronidazole), a lipiarmycin (e.g. fidaxomicin) and a dihydrofolate reductase inhibitor (e.g. a diaminopyrimidine, such as trimethoprim or tetroxoprim). Preferably the antibiotic is selected from a penicillin (such as amoxicillin, ampicillin, piperacillin and flucloxacillin), a polymyxin (such as colistin), a rifamycin (such as rifaximin), a quinolone (such as ciprofloxacin), a sulfonamide (such as sulfomethaxozol), a macrolide (such as erythromycin), a tetracycline (such as tetracycline), an aminoglycoside (such as paromomycin), a cyclic lipopeptide (such as daptomycin), a nitrodimazole (such as metronidazole), and a diaminopyrimidine (such as trimethoprim). In a specific embodiment the antibiotic is selected from amoxicillin, ampicillin, piperacillin, flucloxacillin, colistin, rifaximin, ciprofloxacin, sulfomethaxozol, erythromycin, tetracycline, paromomycin, daptomycin, metronidazole, and trimethoprim. The person skilled in the art will understand that the antibiotic is active in the intestine. One reason for an antibiotic being preferably active in the intestine, without being limited thereto, may be poor absorption from the intestine. Thus, a preferred route of administration of the antibiotic is oral administration.

In certain embodiments the antibiotic may be used in combination, such as in combination with another antibiotic or in combination with another (enhancing) drug. In one embodiment the antibiotic is sulfamethoxazole or trimethoprim or a combination thereof, preferably sulfamethoxazole and trimethoprim. In a preferred embodiment the antibiotic is cotrimoxazol. In yet another embodiment the antibiotic is a penicillin antibiotic, such as amoxicillin, ampicillin, piperacillin or flucloxacillin, in combination with a β-lactamase inhibitor, such as sulbactam or tazobactam. Preferably the antibiotic is ampicillin and piperacillin in combination with a β-lactamase inhibitor. In a preferred embodiment the antibiotic is ampicillin in combination with sulbactam or piperacillin in combination with tazobactam. In cases the Salmonella typhi Ty21a strain comprises an inherent antibiotic resistance or in cases where the DNA molecule comprising at least one eukaryotic expression cassette encoding at least one tumor antigen, stroma antigen and/or checkpoint inhibitor antigen comprises an antibiotic resistance gene the antibiotic may be any antibiotic except the one or more antibiotics the Salmonella typhi Ty21a strain comprising a DNA molecule comprising at least one eukaryotic expression cassette encoding at least one tumor antigen, stroma antigen and/or checkpoint inhibitor antigen is resistant to. In other words, the antibiotic is preferably an antibiotic the Salmonella typhi Ty21a strain comprising a DNA molecule comprising at least one eukaryotic expression cassette encoding at least one tumor antigen, stroma antigen and/or checkpoint inhibitor antigen is not resistant to.

Preferably the antibiotic is administered for at least 3 days, preferably at least one week, more preferably at least 2 weeks.

The cancer to be treated in accordance with the invention is preferably a solid tumor, preferably a solid tumor selected from colorectal cancer, pancreatic cancer, lung cancer, ovarian cancer, mesothelioma, glioblastoma, gastric cancer, hepatocellular cancer, renal cell cancer, prostate cancer, cervical cancer, breast cancer and melanoma. In a preferred embodiment the solid tumor is pancreatic cancer and glioblastoma, more preferably glioblastoma. In one embodiment the cancer is recurrent glioblastoma.

### Combinations Comprising the Salmonella Typhi Ty21a Strain

The Salmonella typhi Ty21a strain for use according to the present invention may also be administered in combination with one or more other compounds or treatments.

In certain embodiments the Salmonella typhi Ty21a strain for use is accompanied by chemotherapy or radiotherapy. The Salmonella typhi Ty21a strain may be administered before, during or after the chemotherapy or the radiotherapy treatment, or before and during the chemotherapy or the radiotherapy treatment. For cure of cancer, complete eradication of cancer stem cells may be essential. Thus, for maximal efficacy, a combination of different therapy approaches may be beneficial.

Chemotherapeutic agents that may be used in combination with the Salmonella typhi Ty21a strain of the present invention may be, for example: gemcitabine, amifostine (ethyol), cabazitaxel, cisplatin, dacarbazine (DTIC), dactinomycin, docetaxel, mechlorethamine, streptozocin, cyclophosphamide, carrnustine (BCNU), lomustine (CCNU), doxorubicin (adriamycin), doxorubicin lipo (doxil), folinic acid, gemcitabine (gemzar), daunorubicin, daunorubicin lipo (daunoxome), procarbazine, ketokonazole, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, oxaliplatin, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

Most preferred chemotherapeutic agents according to the invention are cabazitaxel, carboplatin, oxaliplatin, cisplatin, cyclophosphamide, docetaxel, gemcitabine, doxorubicin, paclitaxel (taxol), irinotecan, vincristine, vinblastine, vinorelbin, folinic acid, 5-fluorouracil and bleomycin, especially gemcitabine.

In certain embodiments the Salmonella typhi Ty21a strain for use is accompanied by biological cancer therapy. In the context of the present invention, the term “biological cancer therapy” refers to cancer therapy involving the use of biopharmaceutical, i.e., a protein based drug (including antibodies) or vaccine, or the use of a cell based treatment, such as CAR-T cells CAR-NK cells or CAR-NKT cells or ex vivo primed antigen presenting cells (APCs).

In a preferred embodiments, the administration of the Salmonella typhi Ty21a strain encoding VEGFR-2 is combined with the administration of the Salmonella typhi Ty21a strain encoding at least one tumor antigen, stroma antigen and/or checkpoint inhibitor antigen selected from the group consisting of WT1, MSLN, CEA, CMV pp65, PD-L1, and FAP, optionally further in combination with at least one checkpoint inhibitor. The Salmonella typhi Ty21a strain encoding VEGFR-2 and the Salmonella typhi Ty21a strain encoding at least one tumor antigen, stroma antigen and/or checkpoint inhibitor antigen selected from the group consisting of WT1, MSLN, CEA, CMV pp65, PD-L1, and FAP may be administered simultaneously or separately.

In the context of the present invention, the term “simultaneously” means administration of the different attenuated strains of Salmonella typhi Ty21a on the same day, more particularly within 12 hours, more particularly within 2 hours. The different attenuated strains of Salmonella typhi Ty21a may be, but do not need to be, in the same dosage form. The term “separately” as used in this context means administration at different days, more particularly at different administration regimens, and in different dosage forms.

In particularly preferred embodiment the Salmonella typhi Ty21a strain is to be administered in combination with at least one checkpoint inhibitor, preferably simultaneously with or prior to said at least one checkpoint inhibitor. The at least one checkpoint inhibitor may be an immunomodulatory antibody, preferably selected from the group consisting of antibodies against PD-1, PD-L1, CTLA-4, IDO, GITR, OX40, TIM-3, LAG-3, KIR, CSF1R and CD137.

According to the invention, the Salmonella typhi Ty21a strain comprising a DNA molecule comprising at least one eukaryotic expression cassette encoding at least one tumor antigen, stroma antigen and/or checkpoint inhibitor antigen (preferably encoding at least one tumor antigen and/or stroma antigen) may further be co-administered with at least one checkpoint inhibitor. The term “checkpoint inhibitor” is used herein synonymously with “immune checkpoint inhibitor”. Typically checkpoint therapy blocks inhibitory checkpoints, restoring immune system function. Specifically the at least one checkpoint inhibitor may be an antibody, particularly selected from a group consisting of antibodies against programmed cell death protein 1 (PD-1), programmed cell death 1 ligand 1 (PD-L1), cytotoxic T-lymphocyte-associated protein 4 (CTLA-4), Indolamin-2,3-Dioxygenase (IDO), glucocorticoid-induced TNFR-related protein (GITR), tumor necrosis factor receptor superfamily, member 4 (OX40), T-cell immunoglobulin and mucin-domain containing-3 (TIM-3), lymphocyte-activation gene 3 (LAG-3), killer-cell immunoglobulin-like receptor (KIR), colony stimulating factor 1 receptor (CSF1R) and CD137. Thus, in a particularly preferred embodiment the Salmonella typhi Ty21a strain is administered in combination with at least one checkpoint inhibitor, wherein the at least one checkpoint inhibitor is preferably an immunomodulatory antibody selected from the group consisting of antibodies against PD-1, PD-L1, CTLA-4, IDO, GITR, OX40, TIM-3, LAG-3, KIR, CSF1R and CD137, preferably an antibody against PD-1, PD-L1 and/or CTLA-4, more preferably an antibody against PD-1 or PD-L1. The checkpoint inhibitor may be administered simultaneously or separately with the at least one Salmonella typhi Ty21a strain comprising a DNA molecule comprising at least one eukaryotic expression cassette encoding at least one tumor antigen, stroma antigen and/or checkpoint inhibitor antigen.

In one embodiment the Salmonella typhi Ty21a strain comprises a DNA molecule comprising at least one eukaryotic expression cassette encoding at least VEGFR-2 for use in the treatment of cancer in a human subject following treatment with an antibiotic, wherein the Salmonella typhi Ty21a strain is to be administered orally and wherein the Salmonella typhi Ty21a strain is administered in combination with at least one checkpoint inhibitor, preferably the at least one checkpoint inhibitor is an immunomodulatory antibody selected from the group consisting of antibodies against PD-1, PD-L1, CTLA-4, IDO, GITR, OX40, TIM-3, LAG-3, KIR, CSF1R and CD137, more preferably an antibody against PD-1, PD-L1 and/or CTLA-4, even more preferably an antibody against PD-1 or PD-L1. The checkpoint inhibitor may be administered simultaneously or separately with the at least one Salmonella typhi Ty21a strain comprising a DNA molecule comprising at least one eukaryotic expression cassette encoding VEGFR-2.

The at least one checkpoint inhibitor, is preferably administered in the approved galenic formulation of the commercial product.

In the context of the present invention, the term “simultaneously” means administration of the attenuated strains of Salmonella typhi Ty21a comprising at least one eukaryotic expression cassette encoding one tumor antigen, stroma antigen and/or checkpoint inhibitor antigen and the checkpoint inhibitor on the same day, more particularly within 12 hours, more particularly within 2 hours. The term “separately” as used in this context means administration at different days, more particularly at different administration regimens, and in different dosage forms.

### Salmonella Typhi Ty21a

Attenuated strains of Salmonella, particularly of the species Salmonella enterica, are attractive vehicles for the delivery of heterologous antigens to the mammalian immune system, since S. enterica strains can potentially be delivered via mucosal routes of immunization, i.e. orally or nasally, which offers advantages of simplicity and safety compared to parenteral administration. Furthermore, Salmonella strains elicit strong humoral and cellular immune responses at the level of both systemic and mucosal compartments. Batch preparation costs are low and formulations of live bacterial vaccines are highly stable. Attenuation can be accomplished by deletion of various genes, including virulence, regulatory, and metabolic genes.

Several Salmonella typhimurium strains attenuated by aro mutations have been shown to be safe and effective delivery vehicles for heterologous antigens in animal models.

According to the invention, the attenuated strain of Salmonella is Salmonella enterica serovar typhi strain Ty21a, also referred to as Salmonella typhi Ty21a. The live, attenuated S. typhi Ty21a strain is the active component of Typhoral L®, also known as Vivotif® (manufactured by Berna Biotech Ltd., a Crucell Company, Switzerland). It is currently the only licensed live oral vaccine against typhoid fever. This vaccine has been extensively tested and has proved to be safe regarding patient toxicity as well as transmission to third parties (Wahdan et al., J. Infectious Diseases 1982, 145:292-295). The vaccine is licensed in more than 40 countries and has been used in millions of individuals including thousands of children for prophylactic vaccination against typhoid fever. It has an unparalleled safety track record. There is no data available indicating that S. typhi Ty21a is able to enter the bloodstream systemically. The live attenuated Salmonella typhi Ty21a vaccine strain thus allows specific targeting of the immune system in the gut, while being safe and well-tolerated. The Marketing Authorization number of Typhoral L® is PL 15747/0001 dated 16 Dec. 1996. One dose of vaccine contains at least 2×10⁹ viable S. typhi Ty21a colony forming units and at least 5×10⁹ non-viable S. typhi Ty21a cells.

This well-tolerated, live oral vaccine against typhoid fever was derived by chemical mutagenesis of the wild-type virulent bacterial isolate S. typhi Ty2 and harbors a loss-of-function mutation in the galE gene resulting in its inability to metabolize galactose. The attenuated bacterial strain is also not able to reduce sulfate to sulfide which differentiates it from the wild-type Salmonella typhi Ty2 strain. With regard to its serological characteristics, the Salmonella typhi Ty21a strain contains the O9-antigen which is a polysaccharide of the outer membrane of the bacteria and lacks the O5-antigen which is in turn a characteristic component of Salmonella typhimurium. This serological characteristic supports the rationale for including the respective test in a panel of identity tests for batch release.

The expression cassette as used in the Salmonella typhi Ty21a strain according to the invention is a eukaryotic expression cassette, particularly comprising a CMV promoter. In the context of the present invention, the term “eukaryotic expression cassette” refers to an expression cassette which allows for expression of the open reading frame in a eukaryotic cell. It has been shown that the amount of heterologous antigen required to induce an adequate immune response may be toxic for the bacterium and may result in cell death, over-attenuation or loss of expression of the heterologous antigen. Using a eukaryotic expression cassette that is not expressed in the bacterial vector but only in the target cell may overcome this toxicity problem and the protein expressed typically exhibits a eukaryotic glycosylation pattern.

A eukaryotic expression cassette comprises regulatory sequences that are able to control the expression of an open reading frame in a eukaryotic cell, preferably a promoter and a polyadenylation signal. Promoters and polyadenylation signals included in the eukaryotic expression cassette comprised by the Salmonella typhi Ty21a strain of the present invention are preferably selected to be functional within the cells of the subject to be immunized. Examples of suitable promoters, especially for the production of a DNA vaccine for humans, include but are not limited to promoters from Cytomegalovirus (CMV), such as the strong CMV immediate early promoter, Simian Virus 40 (SV40), Mouse Mammary Tumor Virus (MMTV), Human Immunodeficiency Virus (HIV), such as the HIV Long Terminal Repeat (LTR) promoter, Moloney virus, Epstein Barr Virus (EBV), and from Rous Sarcoma Virus (RSV), the synthetic CAG promoter composed of the CMV early enhancer element, the promoter, the first exon and the first intron of chicken beta-actin gene and the splice acceptor of the rabbit beta globin gene, as well as promoters from human genes such as human actin, human myosin, human hemoglobin, human muscle creatine, and human metallothionein. In a particular embodiment, the eukaryotic expression cassette contains the CMV promoter. In the context of the present invention, the term “CMV promoter” refers to the strong immediate-early cytomegalovirus promoter.

Examples of suitable polyadenylation signals, especially for the production of a DNA vaccine for humans, include but are not limited to the bovine growth hormone (BGH) polyadenylation site, SV40 polyadenylation signals and LTR polyadenylation signals. In a particular embodiment, the eukaryotic expression cassette comprised by the Salmonella typhi Ty21a strain of the present invention comprises the BGH polyadenylation site.

In addition to the regulatory elements required for expression of the heterologous polypeptide, like a promoter and a polyadenylation signal, other elements can also be included in the eukaryotic expression cassette. Such additional elements include enhancers. The enhancer can be, for example, the enhancer of human actin, human myosin, human hemoglobin, human muscle creatine and viral enhancers such as those from CMV, RSV and EBV.

Regulatory sequences and codons are generally species dependent, so in order to maximize protein production, the regulatory sequences and codons are preferably selected to be effective in the species to be immunized. The person skilled in the art can produce recombinant DNA molecules that are functional in a given subject species, as for example a human subject.

In particular embodiments, the DNA molecule or the DNA molecule comprising the at least one eukaryotic expression cassette comprise an antibiotic resistance gene, such as the kanamycin antibiotic resistance gene, an ori, such as the pMB1 ori or the pUC, and a strong promoter, such as a CMV promoter. In particular embodiments, the recombinant DNA molecule or the DNA molecule comprising the at least one eukaryotic expression cassette is a plasmid, such as a plasmid based on or derived from the commercially available pVAX1™ expression plasmid (Invitrogen, San Diego, California).

This expression vector may be modified by replacing the high copy pUC origin of replication by the low copy pMB1 origin of replication of pBR322. The low copy modification was made in order to reduce the metabolic burden and to render the construct more stable. The generated expression vector backbone was designated pVAX10.

In particular embodiments, the expression plasmid comprises the DNA molecule of SEQ ID NO: 2 (vector backbone pVAX10), which correlates to the sequence of expression vector pVAX10 without the portion of the multiple cloning site which is located between the restriction sites Nhel and Xhol.

The Salmonella typhi Ty21a strain is administered orally. Oral administration is simpler, safer and more comfortable than parenteral administration. However, it has to be noted that the Salmonella typhi Ty21 strain of the present invention may also be administered by any other suitable route. Preferably, a therapeutically effective dose is administered to the subject, and this dose depends on the particular application, the type of malignancy, the subject’s weight, age, sex and state of health, the manner of administration and the formulation, etc. Administration may be single or multiple, as required.

The Salmonella typhi Ty21a strain encoding at least one polypeptide comprising five or more neoantigens may be provided in the form of a solution, a suspension, a lyophilisate, an enteric coated capsule, or any other suitable form. Typically, the Salmonella typhi Ty21a strain is formulated as drinking solution. This embodiment offers the advantage of improved patient compliance. Preferably, the drinking solution comprises means to neutralize gastric acids at least to a certain degree, i.e., to bring the pH of the gastric juice closer to a pH of 7. Preferably, the drinking solution is a buffered suspension comprising the Salmonella typhi Ty21a strain according to the present invention. In a particular embodiment, the buffered suspension is obtained by suspending the Salmonella typhi Ty21a strain in a suitable buffer, preferably containing 2.6 g sodium hydrogen carbonate, 1.7 g L-ascorbic acid, 0.2 g lactose monohydrate and 100 ml of drinking water.

In particular embodiments, a single dose of the Salmonella typhi Ty21a strain comprises from about 10⁶ to about 10⁹, preferably from about 10⁶ to about 10⁸, more preferably from about 10⁶ to about 10⁷ colony forming units (CFU).

More particularly, a single dose of the Salmonella typhi Ty21a strain comprises from about 1×10⁶ to about 1×10⁹, preferably from about 1×10⁶ to about 1×10⁸, more preferably from about 1×10⁶ to about 1×10⁷ colony forming units (CFU).

Furthermore, the Salmonella typhi Ty21a strain according to the invention is preferably administered two to four times in the first week, preferably 4 times in the first week, followed by single dose boosting administration every 2 to 4 weeks, particularly on day 1 and 7, preferably on day 1, 3, 5 and 7 followed by single dose boosting administrations every 2 to 4 weeks.

In this context, the term “about” or “approximately” means within a factor of 3, alternatively within a factor of 2, including within a factor of 1.5 of a given value or range.

It may be favorable dependent on the occurrence of possible side effects, to include treatment with antibiotics or anti-inflammatory agents.

Should adverse events occur that resemble hypersensitivity reactions mediated by histamine, leukotrienes, or cytokines, treatment options for fever, anaphylaxis, blood pressure instability, bronchospasm, and dyspnoea are available. Treatment options in case of unwanted T-cell derived auto-aggression are derived from standard treatment schemes in acute and chronic graft vs. host disease applied after stem cell transplantation. Cyclosporin and glucocorticoids are proposed as treatment options.

In the unlikely case of systemic Salmonella typhi Ty21a type infection, appropriate antibiotic therapy is recommended, for example with fluoroquinolones including ciprofloxacin or ofloxacin. Bacterial infections of the gastrointestinal tract are to be treated with respective agents, such as rifaximin.

### Pharmaceutical Compositions

In a further aspect, the present invention relates to a pharmaceutical composition comprising a Salmonella typhi Ty21a strain comprising a DNA molecule comprising at least one eukaryotic expression cassette encoding at least one tumor antigen, stroma antigen and/or checkpoint inhibitor antigen.

The pharmaceutical composition of the present invention may be in the form of a solution, a suspension, an enteric coated capsule, a lyophilized powder or any other form suitable for the intended oral use. The pharmaceutical composition of the present invention may further comprise one or more pharmaceutically acceptable excipients.

In the context of the present invention, the term “excipient” refers to a natural or synthetic substance formulated alongside the active ingredient of a medication. Suitable excipients include anti-adherents, binders, coatings, disintegrants, flavors, colors, lubricants, glidants, sorbents, preservatives, solvents and sweeteners.

In the context of the present invention, the term “pharmaceutically acceptable” refers to molecular entities and other ingredients of pharmaceutical compositions that are physiologically tolerable and do not typically produce untoward reactions when administered to a mammal (e.g., human). The term “pharmaceutically acceptable” may also mean approved by a regulatory agency of a Federal or a state government or listed in the U.S. Pharmacopeia or other generally recognized pharmacopeia for use in mammals, and, more particularly, in humans.

In particular, suitable drinking solutions typically comprise means to neutralize gastric acids at least to a certain degree, i.e. to bring the pH of the gastric juice closer to a pH of 7. In a particular embodiment, the drinking solution is a buffered suspension obtained by suspending the Salmonella typhi Ty21a strain according to the present invention in a suitable buffer, preferably in a buffer that neutralizes gastric acids to at least a certain degree, preferably in a buffer containing 2.6 g sodium hydrogen carbonate, 1.7 g L-ascorbic acid, 0.2 g lactose monohydrate and 100 ml of drinking water.

In particular embodiments, the pharmaceutical composition is for use as a medicament, particularly for use in the treatment of a cancer (preferably a solid tumor) in a human subject according to the invention. In particular embodiments, the pharmaceutical composition is for use as a medicament, particularly for use in the treatment of cancer (preferably a solid tumor) in a human subject, wherein the subject has been or is treated with at least one antibiotic, preferably following the treatment with an antibiotic.

### EXAMPLES

### Example 1: VXM01 and Avelumab Phase I/II Combination Clinical Trial With Optional Administration of the Antibiotic Cotrim Forte®

The trial is conducted as a multicenter, open-label, PhaseI/II trial (EudraCT.gov no. 2017-003076-31, NCT03750071) to evaluate the efficacy and safety as well as the clinical and immunogenic response of VXM01 in combination with the checkpoint inhibiting antibody avelumab (anti-PD-L1) in patients with non-resectable (n=24) and resectable (n=6) progressive glioblastoma following tumor resection and radiochemotherapy containing temozolomide. 30 patients will be enrolled in 8 study centers in Germany, Netherlands and France. So far 9 non-resectable patients have been enrolled and analysed. In all patients the primary tumor was surgically removed and the patients relapsed under standard therapy, i.e., radiotherapy in combination with temozolomide.

Patients were treated with 10⁶ or 10⁷ CFU of VXM01 and avelumab. Patients were vaccinated orally with VXM01 on day 1, 3, 5, and 7, followed by 4-weekly boosts until progression. Avelumab was administered with a fixed dose of 800 mg intravenously every two weeks until progression. The end of study is week 60. Follow up visits after the end of study were on months 1, 3, 6, 12 and 24 etc. Samples for biomarker and immunogenicity testing were collected at several time points before, during and after treatment.

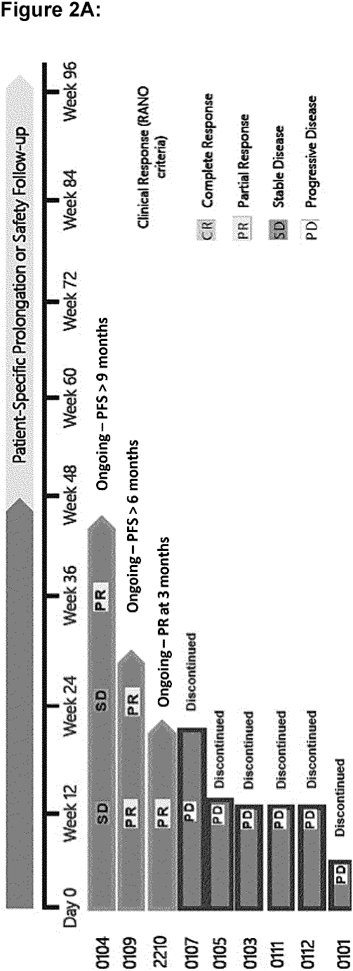

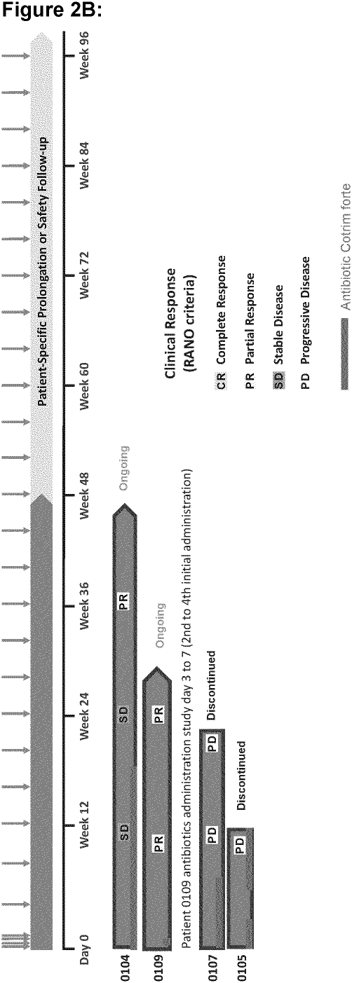

As shown in FIGS. 2A and 3, within the 9 analysed patients, six showed disease progression and three showed partial responses (PR) (patient Nos: 0104, 0109 and 2210), with a tumor reduction of > 50% (FIG. 3). One of the patients with partial response even showed progression-free survival (PFS) for more than 9 months (patient No: 0104). Tumor size has been determined using MRI according to Response Assessment in Neuro-Oncology (RANO) criteria. Two of these patients with partial responses, including the patient that showed progression-free survival for more than 9 months, received the antibiotic Cotrim forte® (sulfamethoxazole 800 mg and trimethoprim 160 mg) at the beginning of the study prior to and at the time of vaccination due to low lymphocyte counts and an associated risk of pneumonia (FIG. 2B). Due to the potential interaction with VXM01 the antibiotic therapy was stopped. In particular, patient No. 0104 received Cotrim forte® from day 0 of this trial for about 20 weeks and patient No. 0109 received Cotrim forte® for about 4 days.

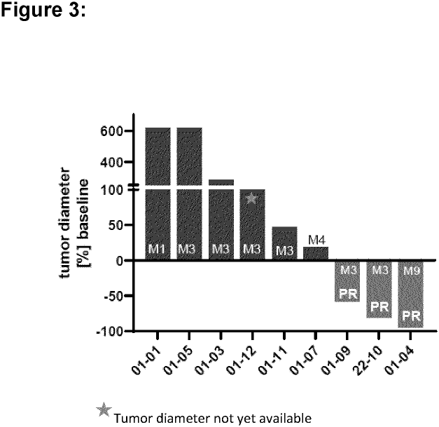

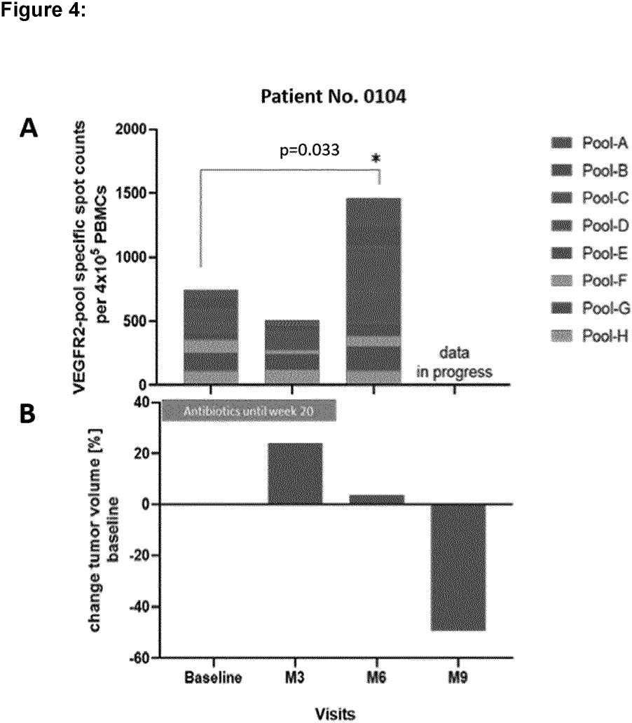

Further, VEGFR-2 specific T cell responses have been analysed in blood samples from patient No: 0104 using an Enzyme Linked Immuno Spot (ELISpot) assay using cryopreserved peripheral blood mononuclear cells at baseline and following 3, 6 and 9 months of treatment. The kinetics of VEGFR-2 specific immune response (FIG. 4A) and tumor response (FIG. 4B) in this patient indicate a major contribution of VXM01 to the therapeutic effect of the treatment. The data demonstrate a clear increase of VEGFR-2 specific immune response after the end of the antibiotics treatment. Moreover, the tumor size decreased following the increase of the VEGFR-2 specific immune response. Intratumoral immune biomarker analysis using tumor tissue immunohistochemistry in sample obtained prior to treatment showed high levels of tumor infiltrating CD8 positive T cells and low levels of Treg cells (FoxP3+ cells) and myeloid-derived suppressor cells (CD68+ cells) (FIG. 5A). Further, no PD1 or PD-L1 expression has been detected in histology sections of tumor samples of patient No: 0104 prior to treatment (FIG. 5B). Overall the data suggest a beneficial effect of antibiotic-pretreatment prior to vaccination with VXM01 alone or in combination with a checkpoint inhibitor such as avelumab.

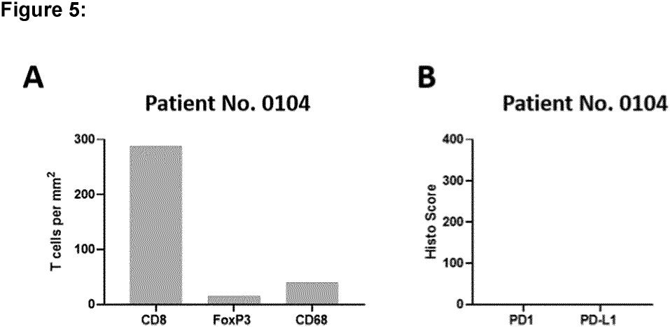

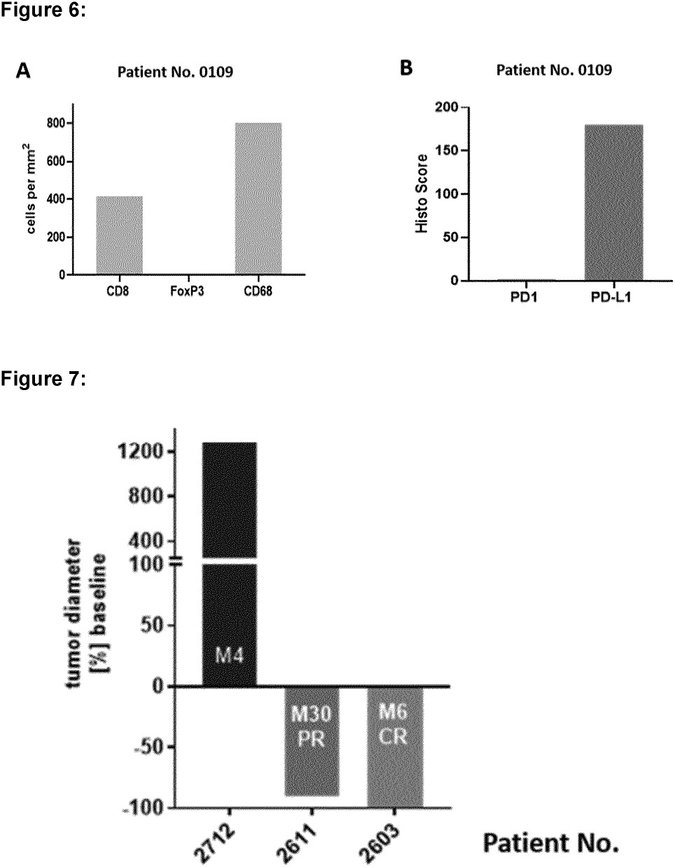

In patient No: 0109 showing a partial response at 3 month (FIGS. 2, 3 and Table 2) intratumoral immune biomarker analysis at baseline showed high levels of tumor infiltrating CD8 positive T cells and MDCS, but no Treg cells (FoxP3+ cells) (FIG. 6A). Although patient No: 0109 has only been treated with an antibiotic for a few days, this may have contributed to the positive outcome in this patient.

Table 2

| | | | | |
|---|---|---|---|---|
| Target Lesions | Target Lesion 1 Tumor Diameter 1 [mm] | Target Lesion 1 Tumor Diameter 2 [mm] | Target Lesion 2 Tumor Diameter 1 [mm] | Target Lesion 2 Tumor Diameter 2 [mm] |
| Baseline | 23 | 10 | 10 | 11 |
| Week 12 | Too small to measure | | Too small to measure | |
| Week 24 | Too small to measure | | Too small to measure | |

### Example 2: VXM01 and Nivolumab Phase I Combination Clinical Trial

Beneficial effects of the use of VXM01 in combination with nivolumab (anti-PD-1) have also been observed in a phase I clinical study in patients with refractory glioblastoma (FIG. 9). Moreover, a synergistic effect of VXM01 and anti-CTLA-4 antibodies has already been reported in mice in WO 2016/202459. Thus, a beneficial effect of antibiotic-pretreatment prior to vaccination with VXM01 is also expected when used in combination with other checkpoint inhibitor such as anti-PD-1 and anti-CTLA-4 antibodies or others.

1. A method for treating cancer in a human subject following treatment with an antibiotic, the method comprising administering a Salmonella typhi Ty21a strain comprising a DNA molecule comprising at least one eukaryotic expression cassette encoding at least one tumor antigen, stroma antigen and/or checkpoint inhibitor antigen , wherein the Salmonella typhi Ty21a strain is to be administered orally.

2. The method of claim 1, wherein the strain Salmonella typhi Ty21a comprises a DNA molecule comprising at least one eukaryotic expression cassette encoding at least one antigen selected from the group consisting of human Wilms' Tumor protein (WT1), human Mesothelin (MSLN), human CEA, CMV pp65, human PD-L1, human VEGFR-2 and human fibroblast activation protein (FAP).

3. The method of claim 1, wherein the Salmonella typhi Ty21a strain comprises a DNA molecule comprising at least one eukaryotic expression cassette encoding at least one neoantigen, preferably at least one eukaryotic expression cassette encoding at least one polypeptide comprising five or more neoantigens.

4. The method of claim 3, wherein the five or more neoantigens are tumor specific antigens identified in the solid tumor of said subject.

5. The method of claim 1, wherein the Salmonella typhi Ty21a strain is administered in combination with at least one checkpoint inhibitor, preferably simultaneously with or prior to said at least one checkpoint inhibitor.

6. The method of claim 5, wherein the at least one checkpoint inhibitor is an immunomodulatory antibody selected from the group consisting of antibodies against PD-1, PD-L1, CTLA-4, IDO, GITR, 0X40, TIM-3, LAG-3, KIR, CSF1R and CD137.

7. The method of claim 1, wherein (a) the Salmonella (a) Ty21a strain is to be administered at least 3 days after completion of the treatment with the antibiotic, and/or (b) the Salmonella typhi Ty21a strain is to be administered within 1 month of completion of the treatment with the antibiotic.

8. The method of claim 1, wherein the antibiotic is an antibiotic the Salmonella typhi Ty21a strain comprising a DNA molecule comprising at least one eukaryotic expression cassette encoding at least one tumor antigen, stroma antigen and/or checkpoint inhibitor antigen is not resistant to.

9. The method claim 1, wherein the antibiotic is a combination preparation.

10. The method of claim 1, wherein the antibiotic is selected from the group consisting of a penicillin, a cephalosporin, a polymyxin, a rifamycin, a lipiarmycin, a quinolone, a sulfonamide, a macrolide, a linocosamide, a tetracycline, an aminoglycoside, a cyclic lipopeptide, a glycylcycline, an oxozolidinone, a nitrodimazole, a lipiarmycin and a dihydrofolate reductase inhibitor.

11. The method of claim 10, wherein the antibiotic is sulfamethoxazole or trimethoprim or a combination thereof.

12. The method of claim 11, wherein the antibiotic is cotrimoxazol.

13. The method of claim 1, wherein the treatment is accompanied by chemotherapy or radiotherapy.

14. The method of claim 1, wherein the cancer is a solid tumor.

15. The method of claim 14, wherein the solid tumor is selected from colorectal cancer, pancreatic cancer, lung cancer, ovarian cancer, mesothelioma, glioblastoma, gastric cancer, hepatocellular cancer, renal cell cancer, prostate cancer, cervical cancer, breast cancer and melanoma.

16. The method of claim 15, wherein the solid tumor is a glioblastoma.

17. The method of claim 16, wherein the solid tumor is recurrent glioblastoma.

18. The method of claim 1, wherein
(a) a single dose of the Salmonella typhi Ty21a strain comprises from about 10⁶ to about 10⁹, more particular from about 10⁶ to about 10⁸, most particular from about 10⁶to about 10⁷ colony forming units (CPU); and/or
(b) wherein the Salmonella typhi Ty21a strain is to be administered 2 to 4 times in the first week, followed by a single dose boosting is to be administration every 2 to 4 weeks.

19. The method of claim 1, wherein the Salmonella typhi Ty21a strain is in the form of a pharmaceutical composition, further comprising at least one pharmaceutically acceptable excipient.


SEQUENCE LISTING (11 sequences)

SEQ ID NO: 1 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 2 - DNA, 3500 bp, Artificial Sequence
  expression plasmid
tgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagacccccatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatc

SEQ ID NO: 3 - protein, 371 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLK

SEQ ID NO: 4 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 5 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 6 - protein, 561 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 7 - protein, 561 aa, Artificial Sequence
  mutated CMV pp65
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 8 - protein, 536 aa, Artificial Sequence
  mutated CMV pp65
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQ

SEQ ID NO: 9 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - protein, 273 aa, Homo sapiens
MFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 11 - protein, 238 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER